(19) United States
(12) Patent Application Publication
(10) Pub. No.: US 2024/0238266 A1
(43) Pub. Date: Jul. 18, 2024

(54) IPTACOPAN FOR THE TREATMENT OF ATYPICAL HEMOLYTIC UREMIC SYNDROME

(71) Applicant: Novartis AG, Basel (CH)

(72) Inventors: Thomas HOLBRO, Basel (CH); Julie Marie-Pomme Gabrielle MILOJEVIC, Village Neuf (FR); Nicholas WEBB, Basel (CH); Anna Svenja SCHUBART WELLENSIEK, Allschwil (CH)

(21) Appl. No.: 18/558,766

(22) PCT Filed: May 6, 2022
(86) PCT No.: PCT/IB2022/054237

§ 371 Date: Nov. 3, 2023

Related U.S. Application Data
(60) Provisional application No. 63/185,742, filed on May 7, 2021.

(51) Int. Cl. A61K 31/454; A61P 13/12

(52) U.S. Cl. CPC A61K 31/454; A61P 13/12

(57) ABSTRACT

Described herein are methods of treating atypical hemolytic uremic syndrome (aHUS) with the Factor B inhibitor LNP023 (iptacopan) or a pharmaceutically acceptable salt thereof, e.g. iptacopan hydrochloride.

### FIELD

The disclosure relates to methods of treating complement driven diseases, and in particular, atypical hemolytic uremic syndrome (aHUS), with the Factor B inhibitor LNP023 (iptacopan) or a pharmaceutically acceptable salt thereof, e.g. iptacopan hydrochloride.

### BACKGROUND

Atypical hemolytic uremic syndrome (aHUS) is an ultra-rare and severe form of thrombotic microangiopathy (TMA) that is characterized by the triad of microangiopathic hemolytic anemia, thrombocytopenia and acute kidney injury (Nester and Thomas (2012) Atypical hemolytic uremic syndrome: what is it, how is it diagnosed, and how is it treated? American Society of Hematology; 617-25). It is caused by uncontrolled activation of the alternative pathway (AP) of the complement system leading to formation and accumulation of the Membrane Attack Complex (MAC, C5b-9) on vascular endothelial cells mainly in the kidneys, ultimately causing kidney endothelial cell damage and local microvascular thrombosis (Nester and Thomas (2012)).

The main affected organs in patients with aHUS are the kidneys, where the microvascular thrombosis leads to kidney insufficiency, including acute kidney injury. Furthermore, aHUS patients present with, inter alia, hemolytic anemia (hemoglobin <10 g/dL), thrombocytopenia (platelets <150×10⁹/L), elevation of serum lactate dehydrogenase (LDH), notable decrease of serum haptoglobin level, and impaired kidney function (serum creatinine >upper limit of normal (ULN) for age); a presentation that is also seen in patients with other forms of TMA (Zhang K, et al. (2017) Atypical Hemolytic Uremic Syndrome: A Brief Review. Hematol Rep; 9:7053; Raina R, et al. (2019) Atypical Hemolytic-Uremic Syndrome: An Update on Pathophysiology, Diagnosis, and Treatment. Ther Apher Dial; 23(1):4-21).

aHUS is an acute disease with many adults and children requiring hemo- or peritoneal dialysis at presentation (Fremeaux-Bacchi V, et al. (2013) Genetics and outcome of atypical hemolytic uremic syndrome: a nationwide French series comparing children and adults. Clin J Am Soc Nephrol; 8:554-62). Most patients present with non-specific clinical symptoms including fatigue, pallor, shortness of breath, and reduced urine output with or without edema (Fakhouri F, et al. (2017) Haemolytic uraemic syndrome. Lancet; 390: 681-96). This varying clinical presentation, combined with the absence of diagnostic biomarkers for aHUS, leads to difficulty in diagnosing aHUS. Therefore, a diagnosis of aHUS remains a clinical one relying on ruling out other forms of TMA (Loirat C, et al. (2016) An international consensus approach to the management of atypical hemolytic uremic syndrome in children. Pediatr Nephrol; 31:15-39), notably thrombotic thrombocytopenia purpura (TTP), as well as hemolytic uremic syndrome (HUS) caused by Shiga toxin-producing Escherichia coli (STEC). Once a suspicion of TMA is established, further testing is done to rule out TTP (through testing of ‘a disintegrin and metalloproteinase with a thrombospondin type 1 motif, member 13’ (ADAMTS13), especially in adults), STX-HUS (through polymerase chain reaction (PCR) based detection, stool culture and blood serology assays), as well as other forms of secondary HUS (due to coexisting diseases such as malignancies or autoimmune diseases including systemic lupus erythematosus (SLE) or drug induced HUS including calcineurin inhibitors). In addition, in very young patients evaluation for streptococcal infection is recommended.

Patients with aHUS have historically had a very poor prognosis, with approximately 36-48% of children and 64-67% of adults reaching end stage kidney disease (kidney failure) or death by 3 to 5 years after onset (Noris M, et al. (2010) Relative role of genetic complement abnormalities in sporadic and familial aHUS and their impact on clinical phenotype. Clin J Am Soc Nephrol; 5:1844-59). The prognosis of aHUS also depends on the underlying genetic or acquired (autoantibody) abnormalities. For example, patients with mutations in FH, FI and anti-FH antibodies have poorer outcomes compared to patients with MCP mutations (Fremeaux-Bacchi, et al. 2013; Schaefer F, et al. (2018) Clinical and genetic predictors of atypical hemolytic uremic syndrome phenotype and outcome. Kidney Int; 94:408-18). The current standard of care (SoC) for patients with aHUS include humanized monoclonal antibodies eculizumab or ravulizumab that inhibit cleavage of C5 to C5a and formation of C5b-9 and prevent terminal complement activation. Treatment with eculizumab substantially improved prognosis for aHUS patients with long-term maintenance of kidney function and low rate of TMA events (Menne J, et al. (2019) Outcomes in patients with atypical hemolytic uremic syndrome treated with eculizumab in a long-term observational study. BMC Nephrol; 20:125). However, treatment with anti-C5 antibody therapy is not available in all countries and, where available, requires intravenous (i.v.) infusions every two (eculizumab) or every eight weeks (ravulizumab) respectively, thus presenting a burden to patients that might affect adherence (Wijnsma K L, et al. (2019) Eculizumab in atypical hemolytic uremic syndrome: strategies toward restrictive use. Pediatr Nephrol; 34:2261-77; Lee H, Kang E, Kang H G, et al. (2020) Consensus regarding diagnosis and management of atypical hemolytic uremic syndrome. Korean J Intern Med; 35(1):25-40).

Plasma exchange and plasma infusions (PE/PI) remain the cornerstone of aHUS treatment in countries where anti-C5 antibody treatment is not available (Loirat C, et al. (2010) Plasmatherapy in atypical hemolytic uremic syndrome. Semin Thromb Hemost; 36(6):673-81). However, its efficacy has never been evaluated in studies (Nester C M, et al. (2015) Atypical aHUS: State of the art. Mol Immunol; 67:31-42). PE/PI works through replacing non-functional proteins and removal of complement FH antibodies (Kavanagh D, et al. (2013) Atypical hemolytic uremic syndrome. Semin Nephrol; 33(6):508-30). However, PE/PI does not address the underlying pathophysiology of aHUS and has limited effects on long-term prognosis for these patients (Raina, et al. 2019). Furthermore, PE/PI pose many challenges, such as adverse effects mainly related to transfusion reactions, access site infections as well as burden to patients with continued therapy (Nester and Thomas 2012). Because FH, FI, FB, and C3 are synthesized in the liver, liver transplantation remains an alternative option for some patients to provide a source of normal protein (Nester and Thomas 2012). Patients reaching kidney failure require kidney transplant or combined liver-kidney transplantation. aHUS patients have high rates of disease recurrence associated with graft loss after kidney transplantation with the outcome being worse in patients with FH and FI mutations (Saland J M, et al. (2009) Liver-kidney transplantation to cure atypical hemolytic uremic syndrome. J Am Soc Nephrol; 20:940-9).

LNP023 (iptacopan) is a novel, orally administered, small molecular weight, first-in-class, selective protease inhibitor that binds to FB (Bb domain) and inhibits C3- (i.e., C3bBb) and C5 (C3bBbC3b) convertases thereby blocking the formation of the MAC. In addition, iptacopan blocks the amplification phase, and halts the complement activation process. In multiple in vitro and in vivo non-clinical mechanistic studies, iptacopan has demonstrated inhibition of the AP (Schubart A, et al. (2019) Small-molecule factor B inhibitor for the treatment of complement-mediated diseases. Proc Natl Acad Sci USA; 116(16):7926-31). Based on the well-characterized AP dysregulation within aHUS pathophysiology, supported by approved terminal complement inhibitor therapies for treatment of aHUS (eculizumab and ravulizumab); an AP pathway inhibitor such as iptacopan is likely to provide therapeutic benefit in aHUS patients.

### SUMMARY

The disclosure relates to methods of treating complement driven diseases, and in particular, atypical hemolytic uremic syndrome (aHUS), with LNP023 (iptacopan) or a pharmaceutically acceptable salt thereof, e.g. iptacopan hydrochloride. Further, the disclosure relates to a Phase 3 clinical study to determine safety and efficacy of iptacopan or a pharmaceutically acceptable salt thereof, e.g., iptacopan hydrochloride, in patients with aHUS. In an embodiment, the patients are treatment naive to complement inhibitor therapy, including anti-C5 antibody therapy. In an embodiment, the patients have been previously treated, or are currently being treated, with complement inhibitor therapy, immunosuppressive therapy, or other therapy prescribed for the treatment of aHUS. Iptacopan belongs to the class of Factor B inhibitors of the complement pathway and acts by inhibiting or suppressing the amplification of the complement system caused by C3 activation irrespective of the initial mechanism of activation.

The disclosure also relates to pharmaceutical compositions, uses, kits, etc. related to iptacopan or a pharmaceutically acceptable salt thereof, e.g., iptacopan hydrochloride.

Iptacopan is chemically designated as 4-((2S,4S)-(4-ethoxy-1-((5-methoxy-7-methyl-1H-indol-4-yl)methyl)piperidin-2-yl))benzoic acid and can be represented by the following chemical structure:

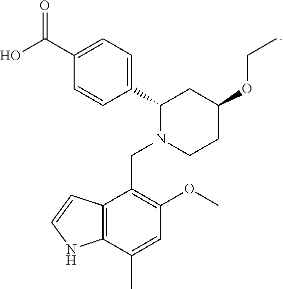 [SMILES: CCO[C@H]1CCN(Cc2c(OC)cc(C)c3[nH]ccc23)[C@H](c2ccc(C(=O)O)cc2)C1]

Iptacopan hydrochloride is chemically designated as 4-((2S,4S)-(4-ethoxy-1-((5-methoxy-7-methyl-1H-indol-4-yl)methyl)piperidin-2-yl))benzoic acid hydrochloride and can be represented by the following chemical structure:

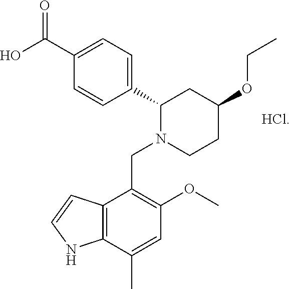 [SMILES: CCO[C@H]1CCN(Cc2c(OC)cc(C)c3[nH]ccc23)[C@H](c2ccc(C(=O)O)cc2)C1]

Iptacopan, iptacopan hydrochloride, and methods of preparation are disclosed in U.S. Pat. Nos. 9,682,968 and 10,093,663 (see Examples 26a, 26c and 26d), which are incorporated herein by reference in their entirety.

The form of iptacopan hydrochloride used as the investigational study drug for this Phase 3 study is a monohydrate (Form HB) as shown in the formula below:

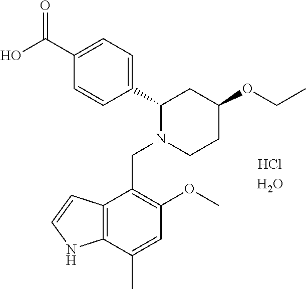 [SMILES: CCO[C@H]1CCN(Cc2c(OC)cc(C)c3[nH]ccc23)[C@H](c2ccc(C(=O)O)cc2)C1]

### (2S,4S)-2-(4-Carboxyphenyl)-4-ethoxy-1-[(5-methoxy-7-methyl-1H-indol-4-yl)methyl]piperidin-1-ium chloride-water (1/1)

Iptacopan hydrochloride monohydrate Form HB and methods for its preparation are disclosed in U.S. Ser. No. 63/026,637 and U.S. Ser. No. 63/052,699, each of which is incorporated herein by reference in its entirety.

We have now devised novel treatments for aHUS patients using LNP023 (iptacopan) or a pharmaceutically acceptable salt thereof, e.g. iptacopan hydrochloride, that are safe and effective and that provide sustained responses for patients. These novel treatments satisfy a long-felt need of clinicians and patients for a safe, sustained, and effective therapy for aHUS.

In one aspect, the disclosure provides a method for treating atypical hemolytic uremic syndrome (aHUS) in a subject, e.g., a patient, in need thereof, the method comprising orally administering to the subject, e.g., patient, iptacopan or a pharmaceutically acceptable salt thereof, e.g., iptacopan hydrochloride, at a therapeutically effective amount, to thereby treat the subject, e.g., patient (wherein the dosing amount refers to the anhydrous free base of iptacopan hydrochloride).

In one aspect, the disclosure provides a method for treating atypical hemolytic uremic syndrome (aHUS) in a subject, e.g., a patient, in need thereof, the method comprising orally administering to the subject, e.g., patient, iptacopan or a pharmaceutically acceptable salt thereof, e.g., iptacopan hydrochloride, at a dose of about 200 mg, to thereby treat the subject, e.g., patient (wherein the dosing amount refers to the anhydrous free base of iptacopan hydrochloride).

In one aspect, the disclosure provides a method for treating atypical hemolytic uremic syndrome (aHUS) in a subject, e.g., a patient, in need thereof, the method comprising orally administering to the subject, e.g., patient, iptacopan or a pharmaceutically acceptable salt thereof, e.g., iptacopan hydrochloride, twice daily (b.i.d.), e.g., about every 12 hours, to thereby treat the subject, e.g., patient (wherein the dosing amount refers to the anhydrous free base of iptacopan hydrochloride).

In another aspect, the disclosure provides a method of assessing the efficacy of treatment in a subject, e.g., patient, treated with, or having been treated with, iptacopan or a pharmaceutically acceptable salt thereof, e.g. iptacopan hydrochloride, at a therapeutically effective amount, e.g., a dose of about 200 mg, e.g., twice daily (b.i.d.), e.g., about every 12 hours, the method comprising assessing the change in the chronic kidney disease (CKD) status in the subject, e.g., patient, to assess the efficacy of treatment (wherein the dosing amount refers to the anhydrous free base of iptacopan hydrochloride).

In another aspect, the disclosure provides a method of assessing the efficacy of treatment in a population of patients treated with, or having been treated with, iptacopan or a pharmaceutically acceptable salt thereof, e.g. iptacopan hydrochloride, at a therapeutically effective amount, e.g., a dose of about 200 mg, e.g., twice daily (b.i.d.), e.g., about every 12 hours, the method comprising determining the percentage of the population of patients achieving complete thrombotic microangiopathy (TMA) response, to assess efficacy of treatment (wherein the dosing amount refers to the anhydrous free base of iptacopan hydrochloride).

In another aspect, the disclosure provides a method of assessing the efficacy of treatment in a population of patients treated with, or having been treated with, iptacopan or a pharmaceutically acceptable salt thereof, e.g. iptacopan hydrochloride, at a therapeutically effective amount, e.g., a dose of about 200 mg, e.g., twice daily (b.i.d.), e.g., about every 12 hours, the method comprising determining the percentage of the population of patients achieving an increase in hemoglobin levels of about 2 g/dL or more, e.g., as compared to baseline, e.g., as compared to hemoglobin levels in the patient population prior to treatment with iptacopan or a pharmaceutically acceptable salt thereof, e.g. iptacopan hydrochloride, to assess efficacy of treatment (wherein the dosing amount refers to the anhydrous free base of iptacopan hydrochloride).

In another aspect, the disclosure provides a method of assessing the efficacy of treatment in a population of patients treated with, or having been treated with, iptacopan or a pharmaceutically acceptable salt thereof, e.g. iptacopan hydrochloride, at a therapeutically effective amount, e.g., a dose of about 200 mg, e.g., twice daily (b.i.d.), e.g., about every 12 hours, the method comprising determining the percentage of the population of patients who no longer require dialysis, to assess efficacy of treatment (wherein the dosing amount refers to the anhydrous free base of iptacopan hydrochloride).

Treatment methods described herein can additionally comprise various evaluation steps prior to and/or following treatment with iptacopan or a pharmaceutically acceptable salt thereof, e.g. iptacopan hydrochloride. In an embodiment, prior to, during, and/or after administration of iptacopan or a pharmaceutically acceptable salt thereof, e.g. iptacopan hydrochloride, the methods further comprise the step of evaluating PK and PD parameters (e.g., plasma concentration of iptacopan or a pharmaceutically acceptable salt thereof, e.g. iptacopan hydrochloride). Evaluation may be achieved by sample analysis of bodily fluid, such as blood or plasma by e.g., mass spectroscopy, e.g. LC-MS.

### BRIEF DESCRIPTION OF THE DRAWINGS

FIG. 1 depicts a schematic of the study design.

FIGS. 2A-2E are tables disclosing the assessment schedule during the core treatment period.

FIG. 3 is a table disclosing the assessment schedule during the extension treatment period.

### DETAILED DESCRIPTION

Described herein are a method of use, and a Phase 3 clinical study to determine safety and efficacy of, iptacopan or a pharmaceutically acceptable salt thereof, e.g., iptacopan hydrochloride, in patients with aHUS. In an embodiment, the patients are treatment naive to complement inhibitor therapy, including anti-C5 antibody therapy. In an embodiment, the patients have been previously treated, or are currently being treated, with complement inhibitor therapy, immunosuppressive therapy, or other therapy prescribed for the treatment of aHUS. The study assesses the effects of iptacopan or a pharmaceutically acceptable salt thereof, e.g., iptacopan hydrochloride, on a range of efficacy assessments relevant to aHUS, including hematological and kidney parameters, dialysis requirement, changes in chronic kidney disease (CKD) stage, as well as patient reported outcomes (PRO) for fatigue (Functional Assessment of Chronic Illness Therapy (FACIT)-fatigue) and quality of life.

In addition, this study will serve as the pivotal trial for the development of iptacopan or a pharmaceutically acceptable salt thereof, e.g., iptacopan hydrochloride, as a treatment for patients with aHUS. Accordingly, described herein are methods of treating aHUS in a patient in need thereof, the method comprising orally administering, e.g., in capsule form, to the patient a twice daily dose, e.g., about every 12 hours, of iptacopan or a pharmaceutically acceptable salt thereof, e.g., iptacopan hydrochloride, (wherein the dosing amount refers to the anhydrous free base of iptacopan hydrochloride). Also described herein are methods of selecting the target patient population, methods of monitoring treatment of the target patient population, and methods of assessing safety and efficacy of treatment of the target patient population.

The details of the disclosure are set forth in the accompanying description below. Although methods and materials similar or equivalent to those described herein can be used in the practice or testing of the present disclosure, illustrative methods and materials are herein described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

Unless specific definitions are provided, the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Remington's Pharmaceutical Sciences,” Mack Publishing Co., Easton, Pa., 21st edition, 2005, which is hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “about” means within +10% of a value.

As used herein, “administering” or “administration” means providing a pharmaceutical agent to an individual, and includes, but is not limited to, administering by a medical professional and self-administering. Administration of a pharmaceutical agent to an individual can be continuous, chronic, short or intermittent.

As used herein, the term “acquire” or “acquiring” as the terms are used herein, refer to obtaining possession of a physical entity (e.g., a sample, e.g., a blood sample or a blood plasma sample), or a value, e.g., a numerical value, by “directly acquiring” or “indirectly acquiring” the physical entity or value. “Directly acquiring” means performing a process (e.g., an analytical method) to obtain the physical entity or value. “Indirectly acquiring” refers to receiving the physical entity or value from another party or source (e.g., a third party laboratory that directly acquired the physical entity or value). Directly acquiring a value includes performing a process that includes a physical change in a sample or another substance, e.g., performing an analytical process which includes a physical change in a substance, e.g., a sample, performing an analytical method, e.g., a method as described herein, e.g., by sample analysis of bodily fluid, such as blood by, e.g., mass spectroscopy, e.g. LC-MS, e.g., LC-MS/MS methods.

As used herein, “dose” means a specified quantity of a pharmaceutical agent provided in a single administration, or in a specified time period. In certain embodiments, a dose can be administered in capsules. As used herein, the dosing amount refers to the anhydrous free base of iptacopan hydrochloride.

As used herein, “individual”, “patient”, “participant”, or “subject” means a human selected for treatment or therapy.

As used herein, “pharmaceutically acceptable salts” means physiologically and pharmaceutically acceptable salts of iptacopan, i.e., salts that retain the desired biological activity of iptacopan and do not impart undesired toxicological effects thereto. The term “pharmaceutically acceptable salt” or “salt” includes a salt prepared from pharmaceutically acceptable non-toxic acids or bases, including inorganic or organic acids and bases. “Pharmaceutically acceptable salts” of iptacopan may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002). Iptacopan hydrochloride and methods for its preparation are disclosed in U.S. Pat. Nos. 9,682,968 and 10,093,663 (see Example 26d), which is incorporated herein by reference in its entirety.

The term “hydrate” as used herein, refers to a crystalline solid where either water is cooperated in or accommodated by the crystal structure e.g. is part of the crystal structure or entrapped into the crystal (water inclusions). Thereby, water can be present in a stoichiometric or non-stoichiometric amount. When water is present in stoichiometric amount, the hydrate may be referred to by adding Greek numeral prefixes. For example, a hydrate may be referred to as a hemihydrate or as a monohydrate depending on the water/compound stoichiometry. The water content can be measured, for example, by Karl-Fischer-Coulometry.

The terms “anhydrous form” or “anhydrate” as used herein refer to a crystalline solid where no water is cooperated in or accommodated by the crystal structure. Anhydrous forms may still contain residual water, which is not part of the crystal structure but may be adsorbed on the surface or absorbed in disordered regions of the crystal. Typically, an anhydrous form does not contain more than 3.0 w-%, e.g., not more than 1.0 w-% of water, based on the weight of the crystalline form.

As used herein, the term “treat” means decrease, suppress, attenuate, diminish, arrest, or stabilize the development or progression of a disorder or disease, e.g., aHUS.

Unless otherwise specified, conventional definitions of terms control and conventional stable atom valences are presumed and achieved in all formulas and groups.

The articles “a” and “an” are used in this disclosure to refer to one or more than one (e.g., to at least one) of the grammatical object of the article. By way of example, “an element” means one element or more than one element.

### Methods of Use

Provided herein is a pharmaceutical composition comprising iptacopan or a pharmaceutically acceptable salt thereof, e.g., iptacopan hydrochloride, for treating atypical hemolytic uremic syndrome (aHUS) in a subject, e.g., a patient, in need thereof, according to the method described herein.

Provided herein is a pharmaceutical composition comprising iptacopan or a pharmaceutically acceptable salt thereof, e.g., iptacopan hydrochloride, for use described herein.

For simplicity, the description below where the term “iptacopan or a pharmaceutically acceptable salt thereof”, “iptacopan hydrochloride”, “iptacopan hydrochloride monohydrate”, or “iptacopan hydrochloride monohydrate Form HB” (collectively referred to as “an iptacopan entity”) may also be substituted with the term “a pharmaceutical composition comprising [any of the aforementioned iptacopan entity]” where appropriate.

Provided herein is a method for treating atypical hemolytic uremic syndrome (aHUS) in a subject, e.g., a patient, in need thereof, the method comprising orally administering to the subject, e.g., patient, iptacopan or a pharmaceutically acceptable salt thereof, e.g., iptacopan hydrochloride, at a therapeutically effective amount, e.g., a dose of about 200 mg, e.g., twice daily (b.i.d.), e.g., about every 12 hours, to thereby treat the subject, e.g., patient (wherein the dosing amount refers to the anhydrous free base of iptacopan hydrochloride).

In another aspect, the disclosure provides use of iptacopan or a pharmaceutically acceptable salt thereof, e.g., iptacopan hydrochloride, in the manufacture of a medicament for the treatment of atypical hemolytic uremic syndrome (aHUS) in a subject, e.g., a patient, wherein the medicament is to be administered orally to the subject, e.g., patient, at a therapeutically effective amount, e.g., a dose of about 200 mg, e.g., twice daily (b.i.d.), e.g., about every 12 hours (wherein the dosing amount refers to the anhydrous free base of iptacopan hydrochloride).

In another aspect, the disclosure provides iptacopan or a pharmaceutically acceptable salt thereof, e.g., iptacopan hydrochloride, for use in the treatment of atypical hemolytic uremic syndrome (aHUS) in a subject, e.g., a patient, wherein the treatment comprises orally administering to the subject, e.g., patient, iptacopan or a pharmaceutically acceptable salt thereof, e.g., iptacopan hydrochloride, at a therapeutically effective amount, e.g., a dose of about 200 mg, e.g., twice daily (b.i.d.), e.g., about every 12 hours (wherein the dosing amount refers to the anhydrous free base of iptacopan hydrochloride).

In another aspect, the disclosure provides use of iptacopan or a pharmaceutically acceptable salt thereof, e.g., iptacopan hydrochloride, in the treatment of atypical hemolytic uremic syndrome (aHUS) in a subject, e.g., a patient, wherein the iptacopan or a pharmaceutically acceptable salt thereof is to be administered orally to the subject, e.g., patient, at a therapeutically effective amount, e.g., a dose of about 200 mg, e.g., twice daily (b.i.d.), e.g., about every 12 hours (wherein the dosing amount refers to the anhydrous free base of iptacopan hydrochloride).

In another aspect, the disclosure provides use of iptacopan or a pharmaceutically acceptable salt thereof, e.g., iptacopan hydrochloride, for the treatment of atypical hemolytic uremic syndrome (aHUS) in a subject, e.g., a patient, wherein the treatment comprises orally administering to the subject, e.g., patient, iptacopan or a pharmaceutically acceptable salt thereof, e.g., iptacopan hydrochloride, at a therapeutically effective amount, e.g., a dose of about 200 mg, e.g., twice daily (b.i.d.), e.g., about every 12 hours (wherein the dosing amount refers to the anhydrous free base of iptacopan hydrochloride).

The aspects of the disclosure above are further illustrated with the embodiments below which may be combined wherever appropriate.

In an embodiment, the treatment comprises orally administering iptacopan hydrochloride to the subject, e.g., patient.

In an embodiment, the treatment comprises orally administering iptacopan hydrochloride monohydrate to the subject, e.g., patient.

In an embodiment, the treatment comprises orally administering iptacopan hydrochloride monohydrate Form HB to the subject, e.g., patient.

In an embodiment, the method comprises orally administering iptacopan hydrochloride to the subject, e.g., patient.

In an embodiment, the method comprises orally administering iptacopan hydrochloride monohydrate to the subject, e.g., patient.

In an embodiment, the method comprises orally administering iptacopan hydrochloride monohydrate Form HB to the subject, e.g., patient.

In an embodiment, the iptacopan or a pharmaceutically acceptable salt thereof to be administered orally is iptacopan hydrochloride.

In an embodiment, the iptacopan or a pharmaceutically acceptable salt thereof to be administered orally is iptacopan hydrochloride monohydrate.

In an embodiment, the iptacopan or a pharmaceutically acceptable salt thereof to be administered orally is iptacopan hydrochloride monohydrate Form HB.

In an embodiment, the medicament to be administered orally comprises iptacopan hydrochloride.

In an embodiment, the medicament to be administered orally comprises iptacopan hydrochloride monohydrate.

In an embodiment, the medicament to be administered orally comprises iptacopan hydrochloride monohydrate Form HB.

In an embodiment, the subject, e.g., patient, is naive to complement inhibitor therapy.

In an embodiment, the subject, e.g., patient, has not been previously treated with, or is not being treated with, an anti-C5 therapy, immunosuppressive therapy (e.g., an immunosuppressive agent, such as, corticosteroids, mycophenolate mofetil (MMF), cyclophosphamide, or rituximab), or other therapy prescribed for aHUS.

In an embodiment, the subject, e.g., patient, has been previously treated, or is currently being treated, with a complement inhibitor therapy.

In an embodiment, the subject, e.g., patient, has been previously treated, or is currently being treated, with an anti-C5 therapy.

In an embodiment, the subject, e.g., patient, has been previously treated, or is currently being treated, with immunosuppressive therapy, or other therapy prescribed for aHUS.

In an embodiment, the anti-C5 therapy is an anti-C5 monoclonal antibody therapy or a biosimilar thereof.

In an embodiment, the anti-C5 therapy is eculizumab or ravulizumab or a biosimilar thereof.

In an embodiment, the subject, e.g., patient, has not been previously treated with, or is not being treated with, plasma exchange or plasma infusions (PE/PI).

In an embodiment, the subject, e.g., patient, has been vaccinated prior to administration of iptacopan or a pharmaceutically acceptable salt thereof, e.g. iptacopan hydrochloride.

In an embodiment, the subject, e.g., patient, has been vaccinated against one or more of Neisseria meningitidis, Streptococcus pneumoniae, and Haemophilus influenzae infections.

In an embodiment, the subject, e.g., patient, has been vaccinated at least one week or at least two weeks, e.g., about 14 days, prior to administration of iptacopan or a pharmaceutically acceptable salt thereof, e.g. iptacopan hydrochloride.

In an embodiment, the subject, e.g., patient, has or is determined to have a genetic mutation associated with aHUS.

In an embodiment, the subject, e.g., patient, has, or is determined to have, a genetic mutation in components regulating the alternative complement pathway.

In an embodiment, the subject, e.g., patient, has, or is determined to have, a genetic mutation selected from the group consisting of C3 (complement component 3), CD46 (cluster of differentiation 46), MCP (membrane cofactor protein), CFB (complement factor B), CFH (complement factor H), CFHR (complement factor H-related protein), and CFI (complement factor I).

In an embodiment, the treatment or treating comprises achieving complete thrombotic microangiopathy (TMA) response.

In an embodiment, the treatment or treating comprises achieving complete thrombotic microangiopathy (TMA) response within 24 weeks, e.g., within 20 weeks, e.g., within 16 weeks, e.g., within 12 weeks, e.g., within 8 weeks, e.g., within 4 weeks, after initiation of orally administering to the subject, e.g., patient.

In an embodiment, achieving complete TMA comprises achieving hematological normalization.

In an embodiment, achieving complete TMA comprises achieving hematological normalization in platelet count.

In an embodiment, achieving complete TMA comprises achieving hematological normalization in platelet count within about 5 weeks, about 4 weeks, about 3 weeks, about 2 weeks, about 1 week, after initiation of orally administering to the subject, e.g., patient.

In an embodiment, achieving hematological normalization in platelet count comprises maintaining platelet count for at least about 1 week, for at least about 2 weeks, for at least about 3 weeks, for at least about 4 weeks, for at least about 5 weeks, for at least about 6 weeks, for at least about 7 weeks, for at least about 8 weeks, for at least about 9 weeks, or for at least about 10 weeks.

In an embodiment, the subject, e.g., patient, has a platelet count (per liter of blood) of less than about 150×10⁹/L prior to treatment with iptacopan or a pharmaceutically acceptable salt thereof, e.g., iptacopan hydrochloride.

In an embodiment, the platelet count (per liter of blood) is normalized after treatment with iptacopan or a pharmaceutically acceptable salt thereof, e.g., iptacopan hydrochloride, to about 150×10⁹/L or more, about 175×10⁹/L or more, about 200×10⁹/L or more, about 225×10⁹/L or more, about 250×10⁹/L or more, about 275×10⁹/L or more, about 300×10⁹/L or more, about 325×10⁹/L or more, about 350×10⁹/L or more, about 375×10⁹/L or more, about 400×10⁹/L or more, about 425×10⁹/L or more, about 450×10⁹/L or more.

In an embodiment, the platelet count (per liter of blood) is normalized after treatment with iptacopan or a pharmaceutically acceptable salt thereof, e.g., iptacopan hydrochloride, to a range of about 150×10⁹/L to about 450×10⁹/L.

In an embodiment, achieving complete TMA comprises achieving normalization of the level of lactate hydrogenase (LDH).

In an embodiment, achieving complete TMA comprises achieving normalization of the level of lactate hydrogenase (LDH) within about 5 weeks, about 4 weeks, about 3 weeks, about 2 weeks, about 1 week, after initiation of orally administering to the subject, e.g., patient.

In an embodiment, achieving hematological normalization in platelet count comprises maintaining the level of lactate hydrogenase (LDH) for at least about 1 week, for at least about 2 weeks, for at least about 3 weeks, for at least about 4 weeks, for at least about 5 weeks, for at least about 6 weeks, for at least about 7 weeks, for at least about 8 weeks, for at least about 9 weeks, or for at least about 10 weeks.

In an embodiment, the subject, e.g., patient, has an LDH level that is about 1.5 times or more of the upper limit of normal (ULN), prior to treatment with iptacopan or a pharmaceutically acceptable salt thereof, e.g. iptacopan hydrochloride, e.g., during the screening period or prior to the start of the screen period.

In an embodiment, achieving complete TMA comprises reducing the level of LDH in the subject, e.g., patient, e.g., as compared to baseline, e.g., as compared to the level of LDH in the subject prior to administration of iptacopan or a pharmaceutically acceptable salt thereof, e.g. iptacopan hydrochloride.

In an embodiment, the LDH level in the subject, e.g., patient, is reduced to below the upper limit of normal.

In an embodiment, the LDH level in the subject, e.g., patient, is reduced by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, e.g., as compared to baseline, e.g., as compared to the level of LDH in the subject prior to administration of iptacopan or a pharmaceutically acceptable salt thereof, e.g. iptacopan hydrochloride.

In an embodiment, the LDH level is reduced by at least about 30%, at least about 35%, at least about 40%, at least about 45%, at least about 50%, at least about 55%, at least about 60%, at least about 65%, or at least about 70%, e.g., as compared to baseline, e.g., as compared to the level of LDH in the subject prior to administration of iptacopan or a pharmaceutically acceptable salt thereof, e.g. iptacopan hydrochloride.

In an embodiment, the LDH level in the subject, e.g., patient, is reduced by at least about 30% or 40%, e.g., as compared to baseline, e.g., as compared to the level of LDH in the subject prior to administration of iptacopan or a pharmaceutically acceptable salt thereof, e.g. iptacopan hydrochloride.

In an embodiment, the LDH level in the subject, e.g., patient, is acquired by sample analysis of a bodily fluid, such as blood or plasma.

In an embodiment, achieving complete TMA response comprises improving kidney function.

In an embodiment, achieving complete TMA response comprises improving kidney function, wherein improving kidney function comprises reducing serum creatinine levels in the subject, e.g., patient.

In an embodiment, achieving complete TMA response comprises improving kidney function, wherein improving kidney function comprises reducing serum creatinine levels in the subject, e.g., patient, within about 16 weeks, about 12 weeks, about 10 weeks, about 8 weeks, about 6 weeks, about 4 weeks, about 3 weeks, about 2 weeks, after initiation of orally administering to the subject, e.g., patient.

In an embodiment, achieving complete TMA response comprises improving kidney function, wherein improving kidney function comprises reducing serum creatinine levels in the subject, e.g., patient, by about 25% or more, e.g., as compared to baseline, e.g., as compared to serum creatinine levels in the subject, e.g., patient, prior to treatment with iptacopan or a pharmaceutically acceptable salt thereof, e.g. iptacopan hydrochloride, within about 12 weeks, about 10 weeks, about 8 weeks, about 6 weeks, about 4 weeks, about 3 weeks, about 2 weeks, after initiation of treatment.

In an embodiment, achieving complete TMA response comprises improving kidney function, wherein improving kidney function comprises reducing serum creatinine levels in the subject, e.g., patient, by about 25% or more, e.g., as compared to baseline, e.g., as compared to serum creatinine levels in the subject, e.g., patient, prior to treatment with iptacopan or a pharmaceutically acceptable salt thereof, e.g. iptacopan hydrochloride; and maintaining the serum creatinine levels for at least for at least about 4 weeks, for at least about 6 weeks, for at least about 8 weeks, for at least about 10 weeks, for at least about 12 weeks, for at least about 16 weeks, or for at least about 20 weeks.

In an embodiment, the subject, e.g., patient, has a serum creatinine level that is about the upper limit of normal (ULN) or more, prior to treatment with iptacopan or a pharmaceutically acceptable salt thereof, e.g. iptacopan hydrochloride, e.g., during the screening period or prior to the start of the screen period.

In an embodiment, the serum creatinine levels are reduced to normal levels.

In an embodiment, the serum creatinine levels are reduced by about 10% or more, by about 15% or more, by about 20% or more, by about 25% or more, by about 30% or more, by about 35% or more, by about 40% or more, by about 45% or more, by about 50% or more, e.g., by about 25% or more, e.g., as compared to baseline, e.g., as compared to serum creatinine levels in the subject, e.g., patient, prior to treatment with iptacopan or a pharmaceutically acceptable salt thereof, e.g. iptacopan hydrochloride.

In an embodiment, the serum creatinine level in the subject, e.g., patient, is acquired by sample analysis.

In an embodiment, the subject, e.g., patient, has a hemoglobin level that is about the lower limit of normal (LLN) or less, prior to treatment or treating with iptacopan or a pharmaceutically acceptable salt thereof, e.g. iptacopan hydrochloride, e.g., during the screening period or prior to the start of the screen period.

In an embodiment, the subject, e.g., patient, has a hemoglobin level that is about 10 g/dL or less, prior to treatment with iptacopan or a pharmaceutically acceptable salt thereof, e.g. iptacopan hydrochloride, e.g., during the screening period or prior to the start of the screen period.

In an embodiment, the hemoglobin level of the subject, e.g., patient, is less than about 9.5 g/dL, less than about 9 g/dL, less than about 8.5 g/dL, less than about 8 g/dL, less than about 7.5 g/dL, less than about 7 g/dL, less than about 6.5 g/dL, less than about 6 g/dL, less than about 5.5 g/dL, or less than about 5 g/dL, prior to treatment with iptacopan or a pharmaceutically acceptable salt thereof, e.g. iptacopan hydrochloride.

In an embodiment, the treatment or treating comprises increasing the hemoglobin level in the subject, e.g., patient, e.g., by about 0.2 g/dL or more, by about 0.3 g/dL or more, by about 0.4 g/dL or more, by about 0.5 g/dL or more, by about 0.75 g/dL or more, by about 1 g/dL or more, by about 1.25 g/dL or more, by about 1.5 g/dL or more, by about 1.75 g/dL or more, by about 2 g/dL or more, by about 2.25 g/dL or more, about 2.5 g/dL or more, by about 2.75 g/dL or more, or about 3 g/dL or more, e.g., as compared to baseline, e.g., as compared to the hemoglobin level in the subject, e.g., patient, prior to treatment or treating with iptacopan or a pharmaceutically acceptable salt thereof, e.g., iptacopan hydrochloride.

In an embodiment, the treatment or treating comprises increasing the hemoglobin level in the subject, e.g., patient, by about 2 g/dL or more, e.g., as compared to baseline, e.g., as compared to the hemoglobin level in the subject, e.g., patient, prior to treatment or treating with iptacopan or a pharmaceutically acceptable salt thereof, e.g., iptacopan hydrochloride.

In an embodiment, the treatment or treating comprises achieving complete TMA response without the use of PE/PI and anti-C5 antibody therapy.

In an embodiment, achieving complete TMA response comprises achieving hematological normalization in platelet count, e.g., ≥150×10⁹/L, and in LDH, e.g., below ULN; and improving kidney function, e.g., ≥25% serum creatinine reduction from baseline.

In an embodiment, efficacy of treatment or treating is determined by comparing a change in hemoglobin level from baseline, e.g., measuring the hemoglobin levels, e.g., at intervals, in the subject, e.g., patient, during and after treatment or treating with iptacopan or a pharmaceutically acceptable salt thereof, e.g., iptacopan hydrochloride, and comparing to baseline, e.g., the hemoglobin level measured prior to treatment or treating with iptacopan or a pharmaceutically acceptable salt thereof, e.g., iptacopan hydrochloride.

In an embodiment, an increase in the hemoglobin level is by about 0.5 g/dL or more, by about 1 g/dL or more, by about 1.5 g/dL or more, about 2 g/dL or more, about 2.5 g/dL or more, about 3 g/dL or more, about 3.5 g/dL or more, about 4 g/dL or more, about 4.5 g/dL or more, or about 5 g/dL or more, e.g., as compared to baseline, e.g., as compared to hemoglobin level in the subject, e.g., patient, prior to treatment or treating with iptacopan or a pharmaceutically acceptable salt thereof, e.g. iptacopan hydrochloride, indicates efficacy of treatment.

In an embodiment, an increase in the hemoglobin level is by about 2 g/dL or more, e.g., as compared to baseline, e.g., as compared to hemoglobin level in the subject, e.g., patient, prior to treatment or treating with iptacopan or a pharmaceutically acceptable salt thereof, e.g. iptacopan hydrochloride, indicates efficacy of treatment.

In an embodiment, the hemoglobin level in the subject, e.g., patient, is acquired by sample analysis.

In an embodiment, the treatment or treating comprises improving kidney function in the subject, e.g., patient, e.g., as compared to baseline, e.g., as compared to the kidney function in the subject, e.g., patient, prior to treatment or treating with iptacopan or a pharmaceutically acceptable salt thereof, e.g. iptacopan hydrochloride.

In an embodiment, the treatment or treating comprises stabilizing or improving the estimated glomerular filtration rate (eGFR).

In an embodiment, improving kidney function is assessed by measuring the eGFR.

In an embodiment, efficacy of treatment or treating is determined by comparing a change in eGFR values from baseline, e.g., measuring eGFR values, e.g., at intervals, in the subject, e.g., patient, during and after treatment or treating with iptacopan or a pharmaceutically acceptable salt thereof, e.g., iptacopan hydrochloride, and comparing to baseline, e.g., the eGFR value measured prior to treatment or treating with iptacopan or a pharmaceutically acceptable salt thereof, e.g., iptacopan hydrochloride.

In an embodiment, the subject, e.g., patient, has been, or is being treated with an immunosuppressive agent, such as a corticosteroid, mycophenolate mofetil (MMF), cyclophosphamide, or rituximab. Additional examples of immunosuppressive agents can be found in Bagga et al. (2019) Pediatric Nephrology 34:1465-1482.

In an embodiment, the subject, e.g., patient, has not been, or is not being treated with an immunosuppressive agent, such as a corticosteroid, mycophenolate mofetil (MMF), cyclophosphamide, or rituximab. In an embodiment, the subject, e.g., patient, has, or is determined to have, antibodies to complement Factor H.

### Patient Selection and Monitoring

The Phase 3 study will enroll aHUS patients diagnosed with aHUS and who are treatment naive to complement inhibitor therapy. Diagnosis of aHUS remains a clinical one relying on ruling out other forms of TMA (Loirat et al. 2016), notably thrombotic thrombocytopeni purpura (TTP), hemolytic uremic syndrome (HUS) caused by Shiga toxin-producing Escherichia coli (STEC-HUS), as well as other forms of secondary HUS (due to coexisting diseases, such as malignancies or autoimmune diseases including systemic lupus erythematosus (SLE) or drug induced HUS including calcineurin inhibitors). In addition, in very young patients evaluation for streptococcal infection is recommended.

Patients will have a diagnosis of aHUS with evidence of TMA including low platelet count (<150×10⁹/L), microangiopathic hemolytic anemia (LDH ≥1.5×ULN, hemoglobin ≤LLN) and decreased kidney function (serum creatinine ≥ULN).

Genetic testing will be done for a selected set of genes known to be involved in aHUS etiology as it provides important prognostic information related to aHUS such as study treatment response, relapse and recurrence after transplantation. However, this aHUS specific genetic analysis will not be part of the screening process or determining eligibility. Additional assessments will include aHUS related biomarkers (such as C3, C4) and autoantibodies to complement proteins (such as factor H autoantibodies). Once clinical diagnosis of aHUS is confirmed by the investigator, genetic and biomarkers/auto-antibody testing will be performed wherever permitted per local regulations and after specific consent has been obtained from the patient.

Iptacopan or a pharmaceutically acceptable salt thereof, e.g., iptacopan hydrochloride, can inhibit complement activation. Accordingly, a subject, e.g., a patient, can be selected for treatment with iptacopan or a pharmaceutically acceptable salt thereof, e.g., iptacopan hydrochloride, by first evaluating the patient to determine whether the subject, e.g., patient, has evidence of TMA, e.g., low platelet count (<150×10⁹/L), microangiopathic hemolytic anemia (LDH ≥1.5×ULN, hemoglobin ≤LLN), and decreased kidney function (serum creatinine ≥ULN), and optionally administering to the subject, e.g., patient, iptacopan or a pharmaceutically acceptable salt thereof, e.g., iptacopan hydrochloride.

In an embodiment, the subject, e.g., patient, can be monitored by evaluating certain PK/PD parameters, such as the level of iptacopan or a pharmaceutically acceptable salt thereof, e.g., iptacopan hydrochloride, the level of LDH, the level of platelets, the level of hemoglobin, or the level of serum creatinine.

### Efficacy Assessment

The primary efficacy assessment is complete TMA response without the use of PE/PI and anti-C5 antibody. Complete TMA response may be assessed by measuring hematological normalization in platelet count, LDH, and improvement in renal function.

Accordingly, provided herein is a method of assessing the efficacy of treatment in a subject, e.g., patient, treated with, or having been treated with, iptacopan or a pharmaceutically acceptable salt thereof, e.g. iptacopan hydrochloride, at a therapeutically effective amount, e.g., a dose of about 200 mg, e.g., twice daily (b.i.d.), e.g., about every 12 hours, the method comprising assessing the change in the chronic kidney disease (CKD) status in the subject, e.g., patient, to assess the efficacy of treatment (wherein the dosing amount refers to the anhydrous free base of iptacopan hydrochloride).

In an embodiment, treatment of the subject, e.g., patient, comprises stabilizing or improving the estimated glomerular filtration rate (eGFR). In an embodiment, efficacy is assessed by measuring the eGFR.

In another aspect, the disclosure provides a method of assessing the efficacy of treatment in a population of patients treated with, or having been treated with, iptacopan or a pharmaceutically acceptable salt thereof, e.g. iptacopan hydrochloride, at a therapeutically effective amount, e.g., a dose of about 200 mg, e.g., twice daily (b.i.d.), e.g., about every 12 hours, the method comprising determining the percentage of the population of patients achieving complete thrombotic microangiopathy (TMA) response, to assess efficacy of treatment (wherein the dosing amount refers to the anhydrous free base of iptacopan hydrochloride).

In an embodiment, the percentage of the population of patients achieving complete TMA response is about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, or about 95% or more.

In an embodiment, the percentage of the population of patients achieving complete TMA response is from about 30% to about 70%.

In an embodiment, achieving complete TMA comprises hematological normalization in platelet count.

In an embodiment, hematological normalization in platelet count comprises maintaining platelet count for at least about 1 week, for at least about 2 weeks, for at least about 3 weeks, for at least about 4 weeks, for at least about 5 weeks, for at least about 6 weeks, for at least about 7 weeks, for at least about 8 weeks, for at least about 9 weeks, or for at least about 10 weeks.

In an embodiment, the platelet count (per liter of blood) is normalized after treatment with iptacopan or a pharmaceutically acceptable salt thereof, e.g., iptacopan hydrochloride, to about 150×10⁹/L or more, about 175×10⁹/L or more, about 200×10⁹/L or more, about 225×10⁹/L or more, about 250×10⁹/L or more, about 275×10⁹/L or more, about 300×10⁹/L or more, about 325×10⁹/L or more, about 350×10⁹/L or more, about 375×10⁹/L or more, about 400×10⁹/L or more, about 425×10⁹/L or more, about 450×10⁹/L or more, e.g., normalized to a range of about 150×10⁹/L to about 450×10⁹/L.

In an embodiment, achieving complete TMA comprises normalization of the level of lactate hydrogenase (LDH).

In an embodiment, the level of LDH is below ULN (upper limit of normal).

In an embodiment, achieving complete TMA comprises improving kidney function in a percentage of the population, e.g., as compared to baseline, e.g., as compared to the kidney function in the patient population prior to treatment with iptacopan or a pharmaceutically acceptable salt thereof, e.g. iptacopan hydrochloride.

In an embodiment, treating the subject, e.g., patient, comprises stabilizing or improving the estimated glomerular filtration rate (eGFR).

In an embodiment, improving kidney function is assessed by measuring the eGFR.

In another aspect, the disclosure provides a method of assessing the efficacy of treatment in a population of patients treated with, or having been treated with, iptacopan or a pharmaceutically acceptable salt thereof, e.g. iptacopan hydrochloride, at a therapeutically effective amount, e.g., a dose of about 200 mg, e.g., twice daily (b.i.d.), e.g., about every 12 hours, the method comprising determining the percentage of the population of patients achieving an increase in hemoglobin levels of about 2 g/dL or more, e.g., as compared to baseline, e.g., as compared to hemoglobin levels in the patient population prior to treatment with iptacopan or a pharmaceutically acceptable salt thereof, e.g. iptacopan hydrochloride, to assess efficacy of treatment (wherein the dosing amount refers to the anhydrous free base of iptacopan hydrochloride).

In an embodiment, the percentage of the population of patients achieving complete TMA response is about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, or about 95% or more.

In another aspect, the disclosure provides a method of assessing the efficacy of treatment in a population of patients treated with, or having been treated with, iptacopan or a pharmaceutically acceptable salt thereof, e.g. iptacopan hydrochloride, at a therapeutically effective amount, e.g., a dose of about 200 mg, e.g., twice daily (b.i.d.), e.g., about every 12 hours, the method comprising determining the percentage of the population of patients who no longer require dialysis, to assess efficacy of treatment (wherein the dosing amount refers to the anhydrous free base of iptacopan hydrochloride).

In an embodiment, the percentage of the population of patients achieving complete TMA response is about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, or about 95% or more.

### SPECIFIC EMBODIMENTS

While various specific embodiments are illustrated and described, it will be appreciated that various changes can be made without departing from the spirit and scope of the disclosure(s). The present disclosure is exemplified by the numbered embodiments set forth below.
1. A method for treating atypical hemolytic uremic syndrome (aHUS) in a subject, e.g., a patient, in need thereof, the method comprising orally administering to the subject, e.g., patient, iptacopan or a pharmaceutically acceptable salt thereof, e.g., iptacopan hydrochloride, a therapeutically effective amount of iptacopan or a pharmaceutically acceptable salt thereof
2. The method of embodiment 1, wherein the therapeutically effective amount is 200 mg (wherein the dosing amount refers to the anhydrous free base of iptacopan).
3. The method of embodiment 1 or 2, wherein the method comprises orally administering to the subject, e.g., patient, twice daily (b.i.d.), iptacopan or a pharmaceutically acceptable salt thereof, e.g., iptacopan hydrochloride.
4. The method of any one of embodiments 1 or 3, wherein the method comprises orally administering to the subject, e.g., patient, about every 12 hours, iptacopan or a pharmaceutically acceptable salt thereof, e.g., iptacopan hydrochloride.
5. The method of any one of embodiments 1 to 4, the method comprising orally administering iptacopan hydrochloride to the subject, e.g., patient.
6. The method of any one of embodiments 1 to 5, the method comprising orally administering iptacopan hydrochloride monohydrate to the subject, e.g., patient.
7. The method of any one of embodiments 1 to 6, the method comprising orally administering iptacopan hydrochloride monohydrate Form HB to the subject, e.g., patient.
8. The method of any one of embodiments 1 to 7, wherein the subject, e.g., patient, is naive to complement inhibitor therapy.
9 The method of any one of embodiments 1 to 8, wherein the subject, e.g., patient, has not been previously treated with, or is not being treated with, an anti-C5 therapy, immunosuppressive therapy (e.g., an immunosuppressive agent, such as, corticosteroids, mycophenolate mofetil (MMF), cyclophosphamide, or rituximab), or other therapy prescribed for aHUS.
10. The method of any one of embodiments s 1 to 9, wherein the subject, e.g., patient, has been previously treated, or is currently being treated, with a complement inhibitor therapy.
11. The method of any one of embodiments 1 to 10, wherein the subject, e.g., patient, has been previously treated, or is currently being treated, with an anti-C5 therapy.
12. The method of any one of embodiments 1 to 11, wherein the subject, e.g., patient, has been previously treated, or is currently being treated, with immunosuppressive therapy (e.g., an immunosuppressive agent, such as, corticosteroids, mycophenolate mofetil (MMF), cyclophosphamide, or rituximab), or other therapy prescribed for aHUS.
13. The method of any one of embodiments 1 to 12, wherein the anti-C5 therapy is an anti-C5 monoclonal antibody therapy or a biosimilar thereof.
14. The method of embodiment 13, wherein the anti-C5 therapy is eculizumab or ravulizumab or a biosimilar thereof.
15. The method of any one of embodiments 1 to 14, wherein the subject, e.g., patient, has not been previously treated with, or is not being treated with, plasma exchange or plasma infusions (PE/PI).
16. The method of any one of embodiments 1 to 15, wherein the subject, e.g., patient, has been vaccinated prior to administration of iptacopan or a pharmaceutically acceptable salt thereof, e.g. iptacopan hydrochloride.
17. The method of embodiment 16, wherein the subject, e.g., patient, has been vaccinated against one or more of Neisseria meningitidis, Streptococcus pneumoniae, and Haemophilus influenzae infections.
18. The method of embodiment 15 or 16, wherein the subject, e.g., patient, has been vaccinated at least one week or at least two weeks, e.g., about 14 days, prior to administration of iptacopan or a pharmaceutically acceptable salt thereof, e.g. iptacopan hydrochloride.
19. The method of any one of embodiments 1 to 18, wherein the subject, e.g., patient, has or is determined to have a genetic mutation associated with aHUS.
20. The method of any one of embodiments 1 to 19, wherein the subject, e.g., patient, has, or is determined to have, a genetic mutation in components regulating the alternative complement pathway.
21. The method of any one of embodiments 1 to 20, wherein the subject, e.g., patient, has, or is determined to have, a genetic mutation selected from the group consisting of C3 (complement component 3), CD46 (cluster of differentiation 46), MCP (membrane cofactor protein), CFB (complement factor B), CFH (complement factor H), CFHR (complement factor H-related protein), CFI (complement factor I), DGKE (diacylglycerol kinase epsilon), and MMACHC (methylmalonic aciduria and homocystinuria type C protein).
22. The method of any one of embodiments 1 to 21, wherein treating the subject, e.g., patient, comprises achieving complete thrombotic microangiopathy (TMA) response.
23. The method of any one of embodiments 1 to 22, wherein treating the subject, e.g., patient, comprises achieving complete thrombotic microangiopathy (TMA) response within 24 weeks, e.g., 20 weeks, e.g., 16 weeks, e.g., within 12 weeks, e.g., within 8 weeks, e.g., within 4 weeks, after initiation of orally administering to the subject, e.g., patient.
24. The method of embodiment 22 or 23, wherein achieving complete TMA response comprises achieving hematological normalization.
25. The method of embodiment 24, wherein achieving hematological normalization comprises achieving hematological normalization in platelet count.
26. The method of embodiment 24, wherein achieving complete TMA comprises achieving hematological normalization in platelet count within about 5 weeks, 4 weeks, 3 weeks, about 2 weeks, about 1 week, after initiation of orally administering to the subject, e.g., patient.
27. The method of any one of embodiments 1 to 27, wherein achieving hematological normalization in platelet count comprises maintaining platelet count for at least about 1 week, for at least about 2 weeks, for at least about 3 weeks, for at least about 4 weeks, for at least about 5 weeks, for at least about 6 weeks, for at least about 7 weeks, for at least about 8 weeks, for at least about 9 weeks, or for at least about 10 weeks.
28. The method of any one of embodiments 1 to 27, wherein the subject, e.g., patient, has a platelet count (per liter of blood) of less than about 150×10⁹/L prior to treatment with iptacopan or a pharmaceutically acceptable salt thereof, e.g., iptacopan hydrochloride.
29. The method of any one of embodiments 1 to 28, wherein the platelet count (per liter of blood) is normalized after treatment with iptacopan or a pharmaceutically acceptable salt thereof, e.g., iptacopan hydrochloride, to about 150×10⁹/L or more, about 175×10⁹/L or more, about 200×10⁹/L or more, about 225×10⁹/L or more, about 250×10⁹/L or more, about 275×10⁹/L or more, about 300×10⁹/L or more, about 325×10⁹/L or more, about 350×10⁹/L or more, about 375×10⁹/L or more, about 400×10⁹/L or more, about 425×10⁹/L or more, about 450×10⁹/L or more, e.g., normalized to a range of about 150×10⁹/L to about 450×10⁹/L.
30. The method of any one of embodiments 1 to 29, wherein achieving complete TMA comprises achieving normalization of the level of lactate hydrogenase (LDH).
31. The method of any one of embodiments 1 to 30, wherein achieving complete TMA comprises achieving normalization of the level of lactate hydrogenase (LDH) within about 5 weeks, 4 weeks, about 3 weeks, about 2 weeks, about 1 week, after initiation of orally administering to the subject, e.g., patient.
32. The method of any one of embodiments 1 to 31, wherein achieving complete TMA comprises achieving normalization of the level of lactate hydrogenase (LDH) within about 5 weeks, 4 weeks, about 3 weeks, about 2 weeks, about 1 week, after initiation of orally administering to the subject, e.g., patient; and maintaining the level of lactate hydrogenase (LDH) for at least about 1 week, for at least about 2 weeks, for at least about 3 weeks, for at least about 4 weeks, for at least about 5 weeks, for at least about 6 weeks, for at least about 7 weeks, for at least about 8 weeks, for at least about 9 weeks, or for at least about 10 weeks.
33. The method of any one of embodiments 1 to 32, wherein the subject, e.g., patient, has an LDH level that is about 1.5 times or more of the upper limit of normal (ULN), prior to treatment with iptacopan or a pharmaceutically acceptable salt thereof, e.g. iptacopan hydrochloride, e.g., during the screening period or prior to the start of the screen period.
34. The method of any one of embodiments 1 to 33, wherein achieving complete TMA comprises reducing the level of LDH in the subject, e.g., patient, e.g., as compared to baseline, e.g., as compared to the level of LDH in the subject prior to administration of iptacopan or a pharmaceutically acceptable salt thereof, e.g. iptacopan hydrochloride.
35. The method of any one of embodiments 1 to 34, wherein the LDH level in the subject, e.g., patient, is reduced by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.
36. The method of any of embodiments 1 to 35, wherein the LDH level is reduced by at least about 30%, at least about 35%, at least about 40%, at least about 45%, at least about 50%, at least about 55%, at least about 60%, at least about 65%, or at least about 70%.
37. The method of any of embodiments 1 to 36, wherein the LDH level is reduced by at least about 30% or 40%, e.g., as compared to baseline, e.g., as compared to the level of LDH in the subject prior to administration of iptacopan or a pharmaceutically acceptable salt thereof, e.g. iptacopan hydrochloride.
38. The method of any one of embodiments 1 to 37, wherein the LDH level is reduced to below the upper limit of normal after initiation of orally administering to the subject, e.g., patient.
39. The method of any one of embodiments 1 to 38, wherein the LDH level in the subject, e.g., patient, is reduced by at least about 30% or 40%.
40. The method of any one of embodiments 1 to 39, wherein the LDH level in the subject, e.g., patient, is acquired by sample analysis of a bodily fluid, such as blood or plasma.
41. The method of any one of embodiments 1 to 40, wherein achieving complete TMA response comprises improving kidney function.
42. The method of embodiment 41, wherein improving kidney function comprises reducing serum creatinine levels in the subject, e.g., patient.
43. The method of embodiment 41 or 42, wherein improving kidney function comprises reducing serum creatinine levels in the subject, e.g., patient, within about 24 weeks, about 20 weeks, about 16 weeks, about 12 weeks, about 10 weeks, about 8 weeks, about 6 weeks, about 4 weeks, about 3 weeks, about 2 weeks, after initiation of orally administering to the subject, e.g., patient.
44. The method of any one of embodiments 1 to 43, wherein improving kidney function comprises reducing serum creatinine levels in the subject, e.g., patient, by about 25% or more, e.g., as compared to baseline, e.g., as compared to serum creatinine levels in the subject, e.g., patient, prior to treatment with iptacopan or a pharmaceutically acceptable salt thereof, e.g. iptacopan hydrochloride, within about 24 weeks, about 20 weeks, about 16 weeks, about 12 weeks, about 10 weeks, about 8 weeks, about 6 weeks, about 4 weeks, about 3 weeks, about 2 weeks, after initiation of treatment.
45. The method of any one of embodiments 1 to 44, wherein improving kidney function comprises reducing serum creatinine levels in the subject, e.g., patient, by about 25% or more, e.g., as compared to baseline, e.g., as compared to serum creatinine levels in the subject, e.g., patient, prior to treatment with iptacopan or a pharmaceutically acceptable salt thereof, e.g. iptacopan hydrochloride; and maintaining the serum creatinine levels for at least for at least about 4 weeks, for at least about 6 weeks, for at least about 8 weeks, for at least about 10 weeks, for at least about 12 weeks, for at least about 16 weeks, or for at least about 20 weeks.
46. The method of any one of embodiments 1 to 45, wherein improving kidney function comprises reducing serum creatinine levels for at least about 1 week, for at least about 2 weeks, for at least about 3 weeks, for at least about 4 weeks, for at least about 5 weeks, for at least about 6 weeks, for at least about 7 weeks, for at least about 8 weeks, for at least about 9 weeks, or for at least about 10 weeks.
47. The method of any one of embodiments 1 to 46, wherein the subject, e.g., patient, has a serum creatinine level that is about the upper limit of normal (ULN) or more, prior to treatment with iptacopan or a pharmaceutically acceptable salt thereof, e.g. iptacopan hydrochloride, e.g., during the screening period or prior to the start of the screen period.
48. The method of any one of embodiments 1 to 47, wherein serum creatinine levels are reduced to normal levels.
49. The method of any one of embodiments 1 to 48, wherein serum creatinine levels are reduced by about 10% or more, by about 15% or more, by about 20% or more, by about 25% or more, by about 30% or more, by about 35% or more, by about 40% or more, by about 45% or more, by about 50% or more, e.g., by about 25% or more, e.g., as compared to baseline, e.g., as compared to serum creatinine levels in the subject, e.g., patient, prior to treatment with iptacopan or a pharmaceutically acceptable salt thereof, e.g. iptacopan hydrochloride.
50. The method of any one of embodiments 1 to 49, wherein the serum creatinine level in the subject, e.g., patient, is acquired by sample analysis.
51. The method of any one of embodiments 1 to 50, wherein the subject, e.g., patient, has a hemoglobin level that is about the lower limit of normal (LLN) or less, prior to treatment with iptacopan or a pharmaceutically acceptable salt thereof, e.g. iptacopan hydrochloride, e.g., during the screening period or prior to the start of the screen period.
52. The method of any one of embodiments 1 to 51, wherein the subject, e.g., patient, has a hemoglobin level that is about 10 g/dL or less, prior to treatment with iptacopan or a pharmaceutically acceptable salt thereof, e.g. iptacopan hydrochloride, e.g., during the screening period or prior to the start of the screen period.
53. The method of any one of embodiments 1 to 52, wherein the hemoglobin level of the subject, e.g., patient, is less than about 9.5 g/dL, less than about 9 g/dL, less than about 8.5 g/dL, less than about 8 g/dL, less than about 7.5 g/dL, less than about 7 g/dL, less than about 6.5 g/dL, less than about 6 g/dL, less than about 5.5 g/dL, or less than about 5 g/dL, prior to treatment with iptacopan or a pharmaceutically acceptable salt thereof, e.g. iptacopan hydrochloride.
54. The method of any one of embodiments 1 to 53, wherein treating the subject, e.g., patient, comprises increasing the hemoglobin level in the subject, e.g., patient, e.g., by about 0.2 g/dL or more, by about 0.3 g/dL or more, by about 0.4 g/dL or more, by about 0.5 g/dL or more, by about 0.75 g/dL or more, by about 1 g/dL or more, by about 1.25 g/dL or more, by about 1.5 g/dL or more, by about 1.75 g/dL or more, by about 2 g/dL or more, by about 2.25 g/dL or more, about 2.5 g/dL or more, by about 2.75 g/dL or more, or about 3 g/dL or more, e.g., about 2 g/dL or more, e.g., as compared to baseline, e.g., as compared to the hemoglobin level in the subject, e.g., patient, prior to treatment with iptacopan or a pharmaceutically acceptable salt thereof, e.g., iptacopan hydrochloride.
55. The method of any one of embodiments 1 to 54, wherein efficacy of treatment is determined by comparing a change in hemoglobin level from baseline, e.g., measuring the hemoglobin levels, e.g., at intervals, in the subject, e.g., patient, during and after treatment with iptacopan or a pharmaceutically acceptable salt thereof, e.g., iptacopan hydrochloride, and comparing to baseline, e.g., the hemoglobin level measured prior to treatment with iptacopan or a pharmaceutically acceptable salt thereof, e.g., iptacopan hydrochloride.
56. The method of embodiment 54 or 55, wherein an increase in the hemoglobin level is by about 0.5 g/dL or more, by about 1 g/dL or more, by about 1.5 g/dL or more, about 2 g/dL or more, about 2.5 g/dL or more, about 3 g/dL or more, about 3.5 g/dL or more, about 4 g/dL or more, about 4.5 g/dL or more, or about 5 g/dL or more, e.g., about 2 g/dL or more, e.g., as compared to baseline, e.g., as compared to hemoglobin level in the subject, e.g., patient, prior to treatment with iptacopan or a pharmaceutically acceptable salt thereof, e.g. iptacopan hydrochloride, indicates efficacy of treatment.
57. The method of any one of embodiments 1 to 56, wherein the hemoglobin level in the subject, e.g., patient, is acquired by sample analysis.
58. The method of any one of embodiments 1 to 57, wherein treating the subject, e.g., patient, comprises improving kidney function in the subject, e.g., patient, e.g., as compared to baseline, e.g., as compared to the kidney function in the subject, e.g., patient, prior to treatment with iptacopan or a pharmaceutically acceptable salt thereof, e.g. iptacopan hydrochloride.
59. The method of any one of embodiments 1 to 58, wherein treating the subject, e.g., patient, comprises stabilizing or improving the estimated glomerular filtration rate (eGFR).
60. The method of claim 59, wherein stabilizing or improving kidney function is assessed by measuring the eGFR.
61. The method of any one of embodiments 1 to 60, wherein efficacy of treatment is determined by comparing a change in eGFR values from baseline, e.g., measuring eGFR values, e.g., at intervals, in the subject, e.g., patient, during and after treatment with iptacopan or a pharmaceutically acceptable salt thereof, e.g., iptacopan hydrochloride, and comparing to baseline, e.g., the eGFR value measured prior to treatment with iptacopan or a pharmaceutically acceptable salt thereof, e.g., iptacopan hydrochloride.
62. The method of any one of embodiments 1 to 61, wherein achieving complete TMA response comprises achieving hematological normalization in platelet count, e.g., ≥150×10⁹/L, and in LDH, e.g., below ULN; and improving kidney function, e.g., ≥25% serum creatinine reduction from baseline.
63. Iptacopan or a pharmaceutically acceptable salt thereof, e.g., iptacopan hydrochloride, for use in the treatment of atypical hemolytic uremic syndrome (aHUS) in a subject, e.g., a patient, wherein the treatment is according to the method of any one of embodiments 1 to 62.
64. A use of iptacopan or a pharmaceutically acceptable salt thereof, e.g., iptacopan hydrochloride, in the treatment of atypical hemolytic uremic syndrome (aHUS) in a subject, e.g., a patient, wherein the treatment is according to the method of any one of embodiments 1 to 62.
65. A use of iptacopan or a pharmaceutically acceptable salt thereof, e.g., iptacopan hydrochloride, for the treatment of atypical hemolytic uremic syndrome (aHUS) in a subject, e.g., a patient, wherein the treatment is according to the method of any one of embodiments 1 to 62.
66. A pharmaceutical composition, comprising iptacopan or a pharmaceutically acceptable salt thereof, e.g., iptacopan hydrochloride, for use for the treatment of atypical hemolytic uremic syndrome (aHUS) in a subject, e.g., a patient, wherein the treatment is according to the method of any one of embodiments 1 to 62.
67. The pharmaceutical composition of embodiment 66, wherein the pharmaceutical composition is for administration given twice daily (b.i.d.), e.g., about every 12 hours.
68. Iptacopan or a pharmaceutically acceptable salt thereof, e.g., iptacopan hydrochloride, for use in the treatment of atypical hemolytic uremic syndrome (aHUS) in a subject, e.g., a patient, wherein the iptacopan or a pharmaceutically acceptable salt thereof is to be administered orally to the subject, e.g., patient, according to any one of embodiments 1 to 62.
69. The iptacopan or a pharmaceutically acceptable salt thereof of embodiment 68, wherein the iptacopan or a pharmaceutically acceptable salt thereof is in a pharmaceutical composition.
70. The iptacopan or a pharmaceutically acceptable salt thereof of embodiment 68 or 69, wherein a subject, e.g., a patient, is naive to complement inhibitor therapy, e.g., an anti-C5 therapy, e.g., an anti-C5 monoclonal antibody therapy or a biosimilar thereof, e.g., eculizumab or ravulizumab or a biosimilar thereof.
71. The iptacopan or a pharmaceutically acceptable salt thereof of any one of embodiment 68 to 70, wherein the patient achieves complete thrombotic microangiopathy (TMA) response, e.g., within 24 weeks, e.g., 20 weeks, e.g., 16 weeks, e.g., within 12 weeks, e.g., within 8 weeks, e.g., within 4 weeks, after initiation of treatment.
72. The iptacopan or a pharmaceutically acceptable salt thereof of any one of embodiment 68 to 71, wherein the patient achieves improvement in kidney function, e.g., comprising reducing serum creatinine levels to normal levels in the subject, e.g., patient, e.g., within 24 weeks, e.g., 20 weeks, e.g., 16 weeks, e.g., within 12 weeks, e.g., within 8 weeks, e.g., within 4 weeks, after initiation of treatment.

### Examples

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof, which may suggest themselves to those skilled in the art without departing from the spirit of the present disclosure and/or scope of the appended claims.

List of Abbreviations

| | |
|---|---|
| (C)FB | (Complement) Factor B |
| (C)FH | (Complement) factor H |
| (C)FI | (Complement) Factor I |
| (e)GFR | (estimated)Glomerular filtration rate |
| (e)PRO | (electronic) Patient Reported Outcome |
| ABMR | antibody-mediated rejection |
| ADAMTS13 | A disintegrin and metalloproteinase with a thrombospondin type 1 motif, |
| ADME | Absorption, distribution, metabolism, and excretion |
| AE | Adverse Event |
| ALP | Alkaline Phosphatase |
| ALT | Alanine Aminotransferase |
| AP | Alternative Pathway |
| AST | Aspartate Aminotransferase |
| b.i.d. | bis in die/twice a day |
| BMT | Bone marrow transplantation |
| C3 | complement component 3 |
| C3G | C3 Glomerulopathy |
| C5 | complement component 5 |
| CI | Confidence Interval |
| CK | Creatine Kinase |
| CKD | chronic kidney disease |
| CKD-EPI | Chronic Kidney Disease Epidemiology Collaboration |
| CMO&PS | Chief Medical Office and Patient Safety |
| CNI | calcineurin inhibitor |
| CO | Country Organization |
| COA | Clinical Outcome Assessment |
| COVID-19 | Coronavirus Disease 2019 |
| CP | Classical pathway |
| CRA | Clinical Research Associate |
| CRF | Case Report/Record Form (paper or electronic) |
| CRO | Contract Research Organization |
| CRP | C Reactive protein |
| CSR | Clinical study report |
| CT | Computed tomography |
| CV | coefficient of variation |
| DGKE | Diacylglycerol kinase ε |
| DHT | dihydrotestosterone |
| DIC | Disseminated Intravascular Coagulation |
| DMC | Data Monitoring Committee |
| ECG | Electrocardiogram |
| EDC | Electronic Data Capture |
| EMA | European Medicines Agency |
| EoS | End of Study |
| EoT | End of Treatment |
| FACIT | Functional Assessment of Chronic Illness Therapy |
| FAS | Full Analysis Set |
| FDA | Food and Drug Administration |
| FHAA | Antibodies to complement factor H protein |
| FIH | First in Human |
| FSH | follicle stimulating hormone |
| G6PD | glucose-6-phosphate dehydrogenase |
| GCP | Good Clinical Practice |
| GGT | Gamma-glutamyl transferase |
| GLMM | General Linear Mixed model |
| h | Hour |
| HBsAg | Hepatitis B surface antigen |
| HCV | Hepatitis C Virus |
| HIV | Human immunodeficiency virus |
| HRQoL | Health-Related Quality of Life |
| HRU | Healthcare Resource Utilization |
| HSCT | Hematopoietic stem cell transplantation |
| i.v. | intravenous |
| IA | Interim analysis |
| IB | Investigator's Brochure |
| ICF | Informed Consent Form |
| ICH | International Council for Harmonization of Technical Requirements for |
| IEC | Independent Ethics Committee |
| IgAN | IgA nephropathy |
| iMN | idiopathic membranous nephropathy |
| IMP | Investigational Medicinal Product |
| IN | Investigator Notification |
| INR | International Normalized Ratio |
| IRB | Institutional Review Board |
| IRT | Interactive Response Technology |
| IUD | intrauterine device |
| IUS | intrauterine system |
| IVIg | intravenous immunoglobulin |
| LC-MS/MS | Liquid Chromatography with tandem mass spectrometry |
| LDH | lactate dehydrogenase |
| LDL | Low density lipoprotein |
| LFT | Liver function test |
| LH | luteinizing hormone |
| LLN | lower limit of normal |
| LLOQ | lower limit of quantification |
| LOAEL | Lowest observed adverse effect level |
| MAC | Membrane Attack Complex (C5b-9) |
| MCP | membrane cofactor protein |
| MCS | Mental Component Summary |
| MedDRA | Medical dictionary for regulatory activities |
| mg | milligram(s) |
| mL | milliliter(s) |
| MMACHC | methylmalonic aciduria and homocystinuria type C protein |
| MMF | mycophenolate mofetil |
| MMRM | Mixed Model of Repeated Measures |
| MRI | magnetic resonance imaging |
| ms | Millisecond |
| mTORI | rapamycin inhibitor |
| NTI | narrow therapeutic index |
| NYHA | New York Heart association |
| OATP | organic anion-transporting polypeptide |
| P-gp | Permeability glycoprotein |
| PCS | Physical component summary |
| PD | Pharmacodynamic(s) |
| PE | Plasma exchange |
| PGIS | Patient Global Impression of Severity |
| PI | Plasma infusion |
| PK | Pharmacokinetic(s) |
| PNH | Paroxysmal Nocturnal Hemoglobinuria |
| PRO | Patient Reported Outcomes |
| PT | prothrombin time |
| PTH | Parathyroid hormone (parathormone or parathyrin) |
| QMS | Quality Management System |
| QTcF | QT interval corrected by Fridericia's formula |
| REP | Rollover Extension Program |
| SAE | Serious Adverse Event |
| SAP | Statistical Analysis Plan |
| SD | standard deviation |
| SF-36 v2 | Short-form 36 health survey questionnaire version 2 |
| SLE | systemic lupus erythematosus |
| SoC | Standard of Care |
| STEC | Shiga toxin-producing Escherichia coli |
| STX-HUS | Shiga toxin-related hemolytic uremic syndrome |
| SUSAR | Suspected Unexpected Serious Adverse Reaction |
| TBL | Total Bilirubin |
| TCMR | T-Cell mediated rejection |
| TMA | Thrombotic microangiopathy |
| TSH | thyroid stimulating hormone |
| TTP | Thrombotic Thrombocytopenic Purpura |
| ULN | upper limit of normal |
| ULOQ | Upper limit of quantification |
| US | Ultrasound |
| VAS | visual analogue scale |
| WHO | World Health Organization |
| WoC | Withdrawal of (study) Consent |

Glossary of Terms

| | |
|---|---|
| Additional | Medicinal products that may be used during the clinical trial as |
| treatment/Other | described in the protocol, but not as an investigational medicinal |
| treatment | product (e.g. any background therapy), also include treatment |
| | that may be needed/allowed during the conduct of the study (i.e. |
| | concomitant or rescue therapy) |
| Assessment | A procedure used to generate data required by the study |
| Biologic Samples | A biological specimen including, for example, blood (plasma, |
| | serum), saliva, tissue, urine, stool, etc. taken from a study |
| | participant |
| Clinical Outcome | A measure that describes or reflects how a participant feels, |
| Assessment | functions, or survives |
| (COA) | |
| Coded Data | Personal Data which has been de-identified by the investigative |
| | center team by replacing personal identifiers with a code. |
| Cohort | A group of individuals who share a common exposure, |
| | experience or characteristic, or a group of individuals followed- |
| | up or traced over time |
| Discontinuation | Point/time when the participant permanently stops receiving the |
| from study | study treatment and further protocol required assessments or |
| | follow-up, for any reason. No specific request is made to stop |
| | the use of their samples or data. |
| Discontinuation | Point/time when the participant permanently stops receiving the |
| from study | study treatment for any reason (prior to the planned completion |
| treatment | of study drug administration, if any). Participant agrees to the |
| | other protocol required assessments including follow-up. No |
| | specific request is made to stop the use of their samples or data. |
| Dosage | Dose of the study treatment given to the participant in a time |
| | unit (e.g. 100 mg once a day, 75 mg twice a day) |
| Electronic Data | Electronic data capture (EDC) is the electronic acquisition of |
| Capture (EDC) | clinical study data using data collection systems, such as Web- |
| | based applications, interactive voice response systems and |
| | clinical laboratory interfaces. EDC includes the use of electronic |
| | Case Report Forms which are used to capture data transcribed |
| | from source data/documents used at the point of care |
| End of the clinical trial | The end of the clinical trial is defined as the last visit of |
| | the last participant |
| Enrollment | Point/time of participant entry into the study at which informed |
| | consent must be obtained. The action of enrolling one or more |
| | participants |
| Estimand | As defined in the ICH E9(R1) addendum, estimand is a precise |
| | description of the treatment effect reflecting the clinical question |
| | posed by the trial objective. It summarizes at a population-level |
| | what the outcomes would be in the same participants under |
| | different treatment conditions being compared. Attributes of an |
| | estimand include the population, variable (or endpoint) and |
| | treatment of interest, as well as the specification of how the |
| | remaining intercurrent events are addressed and a population |
| | level summary for the variable |
| Intercurrent events | Events occurring after treatment initiation that affect either the |
| | interpretation or the existence of the measurements associated |
| | with the clinical question of interest |
| Investigational study | The drug whose properties are being tested in the study |
| drug/treatment | |
| Medication number | A unique identifier on the label of medication kits |
| Mis-randomized | Mis-randomized participants are those who were not qualified for |
| participants | randomization and who did not take study treatment, but have |
| | been inadvertently randomized into the study or the participant |
| | allocated to an invalid stratification factor. |
| Off-site | Describes trial activities that are performed at remote location by |
| | an off-site healthcare professional, such as procedures performed |
| | at the participant's home. |
| Off-site healthcare | A qualified healthcare professional, such as include those used in |
| Professional (OHP) | the study e.g. Nurse, Phlebotomist, Physician, who performs |
| | certain protocol procedures for the participant in an off-site |
| | location such as a participant's home. |
| Other treatment | Treatment that may be needed/allowed during the conduct of the |
| | study (i.e. concomitant or rescue therapy) |
| Participant | A trial participant (can be a healthy volunteer or a patient). |
| | “Participant” terminology is used in the protocol whereas term |
| | “Subject” is used in data collection. |
| Participant number | A unique number assigned to each participant upon signing the |
| | informed consent. This number is the definitive, unique identifier |
| | for the participant and should be used to identify the participant |
| | throughout the study for all data collected, sample labels, etc. |
| Patient-Reported | A measurement based on a report that comes directly from the |
| Outcome (PRO) | patient about the status of a participant's health condition without |
| | amendment or interpretation of the patient's report by a clinician |
| | or anyone else |
| Period | The subdivisions of the trial design (e.g. Screening, Treatment, |
| | Follow- up) which are described in the Protocol. Periods define |
| | the study phases and will be used in clinical trial database setup |
| | and eventually in analysis |
| Personal data | Participant information collected by the Investigator that is coded |
| | and transferred to Novartis for the purpose of the clinical trial. |
| | This data includes participant identifier information, study |
| | information and biological samples. |
| Re-screening | If a participant fails the initial screening and is considered as a |
| | Screen Failure, he/she can be invited once for a new Screening |
| | visit after medical judgment and as specified by the protocol |
| Remote | Describes any trial activities performed at a location that is not the |
| | investigative site where the investigator will conduct the trial, but |
| | is for example a home or another appropriate location |
| Screen Failure | A participant who did not meet one or more criteria that were |
| | required for participation in the study |
| Source Data/Document | Source data refers to the initial record, document, or primary |
| | location from where data comes. The data source can be a |
| | database, a dataset, a spreadsheet or even hard-coded data, such as |
| | paper or eSource |
| Start of the clinical trial | The start of the clinical trial is defined as the signature of the |
| | informed consent by the first participant |
| Study treatment | Any drug or combination of drugs or intervention administered to |
| | the study participants as part of the required study procedures; |
| | includes investigational drug(s), control(s) or background therapy |
| Study treatment | When the participant permanently stops taking any of the study |
| discontinuation | drug(s) prior to the defined study treatment completion date (if |
| | any) for any reason; may or may not also be the point/time of |
| | study discontinuation |
| Tele-visit | Procedures or communications conducted using technology such |
| | as telephone or video-conference, whereby the participant is not at |
| | the investigative site where the investigator will conduct the trial. |
| Treatment of interest | The treatment of interest and, as appropriate, the alternative |
| | treatment to which comparison will be made. These might be |
| | individual interventions, combinations of interventions |
| | administered concurrently, |
| | e.g. as add-on to standard of care, or might consist of an overall |
| | regimen involving a complex sequence of interventions. This is |
| | the treatment of interest used in describing the related clinical |
| | question of interest, which might or might not be the same as the |
| | study treatment. |
| Variable (or endpoint) | The variable (or endpoint) to be obtained for each participant that |
| | is required to address the clinical question. The specification of |
| | the variable might include whether the participant experiences an |
| | intercurrent event. |
| Withdrawal of study | Withdrawal of consent from the study occurs when the participant |
| consent (WoC)/ | explicitly requests to stop use of their data and biological samples |
| Opposition to use of | (opposition to use data and biological samples) AND no longer |
| data/ | wishes to receive study treatment, AND does not agree to further |
| biological samples | protocol required assessments. This request should be in writing |
| | (depending on local regulations) and recorded in the source |
| | documentation. |
| | Opposition to use data/biological samples occurs in the countries |
| | where collection and processing of personal data is justified by a |
| | different legal reason than consent. |

### Example 1. A Multicenter, Single-Arm, Open Label Study to Evaluate Efficacy and Safety of LNP023 (Iptacopan) Hydrochloride, Administered Oral, Twice Daily in Adult Patients with aHUS Who are Treatment Naive to Complement Inhibitor Therapy, Including Anti-C5 Antibody

### Purpose

This trial is a multicenter, single-arm, open label study with a 26-week core treatment period followed by a 26-week Extension Treatment period. The purpose of this Phase 3 study is to evaluate the efficacy and safety of LNP023 (iptacopan) treatment given as 200 mg twice daily (wherein the dosing amount refers to the anhydrous free base of iptacopan) in adult patients with aHUS who are treatment naive to complement inhibitor therapy, including anti-C5 antibody. The study will assess the effects of study treatment iptacopan on a range of efficacy assessments relevant to aHUS including hematological and kidney parameters, dialysis requirement, changes in chronic kidney disease (CKD) stage, as well as patient reported outcomes (PRO) for fatigue (Functional Assessment of Chronic Illness Therapy (FACIT)-fatigue) and quality of life. This study will serve as the pivotal trial for the development of iptacopan as a treatment for patients with aHUS. Data from the 26-week core treatment period (Day 182) of this study will provide the pivotal efficacy and safety data. The 26-week Extension Treatment phase (Day 364) will provide further long-term safety and efficacy data on iptacopan in aHUS patients.

### Investigational Study Drug

The form of the investigational study drug, iptacopan hydrochloride, chosen for this Phase 3 study is a monohydrate Form HB as shown in the formula below:

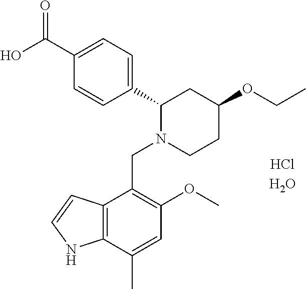 [SMILES: CCO[C@H]1CCN(Cc2c(OC)cc(C)c3[nH]ccc23)[C@H](c2ccc(C(=O)O)cc2)C1]

### (2S,4S)-2-(4-Carboxyphenyl)-4-ethoxy-1-[(5-methoxy-7-methyl-1H-indol-4-yl)methyl]piperidin-1-ium chloride-water (1/1)

Accordingly, in Example 1, “iptacopan” means iptacopan hydrochloride monohydrate Form HB. Iptacopan hydrochloride monohydrate Form HB and methods for its preparation are disclosed in U.S. Ser. No. 63/026,637 and U.S. Ser. No. 63/052,699, each of which is incorporated herein by reference in its entirety.

### Primary Objectives and Endpoints

The primary objective is to assess the proportion of participants treated with iptacopan achieving complete thrombotic microangiopathy (TMA) response during 26 weeks of study treatment. The endpoint is determined by a complete TMA response without the use of PE/PI and anti-C5 antibody during 26 weeks of study treatment. Complete TMA response is defined as (1) hematological normalization in platelet count (platelet count ≥150×10⁹/L) and LDH (below ULN), and (2) improvement in kidney function (≥25% serum creatinine reduction from baseline), maintained for two measurements obtained at least four weeks apart, and any measurement in between.

### Secondary Objectives and Endpoints/Estimands

The secondary objectives are to:
· To assess the effect of treatment with iptacopan on time to complete TMA response, where the endpoint is determined by the time to achieve TMA response during 26 weeks of study treatment being the relevant endpoint;
· To assess the proportion of participants achieving an increase from baseline in hemoglobin levels of ≥2 g/dL, where the endpoint, i.e., response, is determined by an increase in hemoglobin of ≥2 g/dL from baseline, observed at two measurements obtained at least 4 weeks apart and any measurement in between during 26 weeks of study treatment;
· To assess the effect of iptacopan study treatment on hematologic parameters (platelets, LDH, hemoglobin), where the endpoint is determined by the change from baseline in hematologic parameters (platelets, LDH, hemoglobin) at Week 26;
· To assess the effect of iptacopan study treatment on dialysis requirement status, where the endpoint is determined by the proportion of participants on dialysis (done for this TMA event) who no longer require dialysis through 26 weeks of study treatment;
· To assess the effect of iptacopan study treatment on estimated glomerular filtration rate (eGFR), where the endpoint is determined by change from baseline in eGFR values at Week 26; and
· To assess the effect of iptacopan study treatment on chronic kidney disease (CKD) stage, where the endpoint is determined by the change from baseline in CKD stage (1-5) based on eGFR categories at Week 26.

Additional objectives include:
· To assess the effect of iptacopan study treatment on patient-reported overall fatigue severity and health-related quality of life, where the endpoint is determined by the change from baseline in patient-reported outcomes score for FACIT-Fatigue, Patient Global Impression of Severity (PGIS), EuroQol 5-level EQ-5D version (EQ-5D-5L) and Short-form 36 health survey questionnaire version 2 (SF-36 v2) at Week 26; and
· To assess safety and tolerability of iptacopan study treatment, where the endpoint is determined by the safety evaluations (including adverse events/serious adverse events, safety laboratory parameters and vital signs)

The primary estimand is the complete TMA response without the use of PE/PI and anti-C5 antibody during 26 weeks of study treatment and is defined as (1) hematological normalization in platelet count (platelet count ≥150×10⁹/L) and LDH (below ULN), and (2) improvement in renal function (≥25% serum creatinine reduction from baseline), maintained for two measurements obtained at least four weeks apart and any measurements in between.

For secondary estimands, the strategy is described with each secondary endpoint. In general, for time to TMA response endpoint, it will apply the same estimand framework as the primary endpoint. For change from baseline endpoints, the treatment policy strategy will be applied. For participants with an increase in hemoglobin ≥2 g/dL from baseline, a composite strategy will be applied.

### Exploratory Objectives

The exploratory objectives are to:
· To characterize the PK of iptacopan study treatment in the aHUS population;
· To assess the effect of iptacopan study treatment on the utilization of Health Care resources;
· To assess the effects of iptacopan study treatment on aHUS sign and symptoms;
· To explore the effect of iptacopan study treatment in aHUS patients with known genetic mutations; and
· To explore blood and/or urine biomarkers related to treatment effect with iptacopan study treatment.

### Study Design

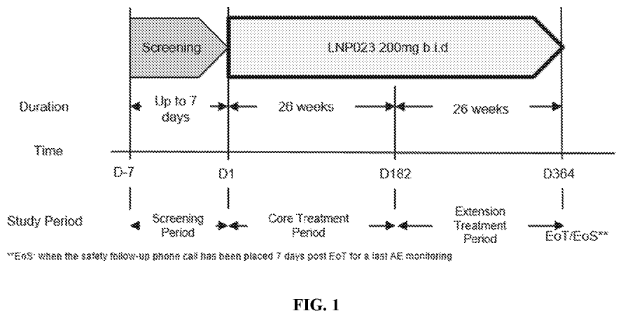

This Phase 3 study is a multicenter, single arm, open-label trial in adult patients diagnosed with aHUS and who are treatment naive to complement inhibitor therapy. The study will consist of three periods as showing in FIG. 1.
· A screening period lasting up to 7 days
· A 26-week single arm, open-label core treatment period for the primary efficacy and safety analysis
· A 26-week open-label, Extension Treatment period to assess long-term safety, tolerability and efficacy of iptacopan

The total study treatment duration spans over 52 weeks. The study will ensure approximately 50 adult patients are treated with iptacopan. Study participants are adult patients diagnosed with aHUS, treatment naive to complement inhibitor therapy, and with evidence of TMA including:
· low platelet count (<150×10⁹/L)
· microangiopathic hemolytic anemia (LDH ≥1.5×ULN, hemoglobin slower limit of normal (LLN))
· decreased kidney function (serum creatinine ≥ULN in adults (≥18 years of age) (patients requiring dialysis for acute kidney injury are eligible).
   The study will aim for approximately 5 patients treated with iptacopan with prior kidney transplantation.

An interim analysis (IA) will be performed at the time when approximately 8 adult participants have completed 12 weeks of study treatment (Day 84 visit). The intent of this IA is to provide preliminary evidence of efficacy and safety of iptacopan in treatment naive aHUS patients. The IA will include analyses of the primary endpoint (complete TMA response) at 12 weeks and its components [hematological normalization (platelet count and LDH), improvement in kidney function (≥25% serum creatinine reduction from baseline)] as well as hematological parameters (platelets, LDH, hemoglobin) and kidney outcomes (GFR and dialysis requirement) relevant to clinical benefit in patients with aHUS. In addition, safety endpoints (including vital signs, safety labs, adverse events, serious adverse events, discontinuations etc.) will be reviewed.

### Study Design Rationale

This Phase 3 study is designed as a multicenter, single-arm, open label trial to study efficacy and safety of oral, twice daily iptacopan in adult aHUS patients, who are naive to complement inhibitor therapy including anti-C5 antibody. A single arm design has been chosen for this study for the following reasons:
· A placebo-controlled design is deemed unethical because aHUS is a severe, ultra-rare, rapidly progressing disease requiring early treatment. Moreover, in countries where SoC (eculizumab or ravulizumab) is available, a placebo arm will not be justified.
· The single arm, open-label design is widely used in rare diseases due to challenges associated with conducting studies in these patient populations (Bell S A, et al. (2014) A comparison of interventional clinical trials in rare versus non-rare diseases: an analysis of ClinicalTrials.gov. Orphanet J Rare Dis; 9:170) as a randomized controlled study (vs. SoC) would require a large sample size. Due to the ultra-rare nature of the disease and the challenge recruiting this patient population in a reasonable timeframe, a single arm trial design will allow earlier availability of data for a potentially beneficial treatment for patients with aHUS.
· All of the approved therapies in aHUS patients have adopted a similar single arm open label design for their pivotal studies (Fakhouri F, et al. (2016) Terminal Complement Inhibitor Eculizumab in Adult Patients With Atypical Hemolytic Uremic Syndrome: A Single-Arm, Open-Label Trial. Am J Kidney Dis; 68(1):84-93; Rondeau E, et al. (2020) The long-acting C5 inhibitor, Ravulizumab, is effective and safe in adult patients with atypical hemolytic uremic syndrome naïve to complement inhibitor treatment. Kidney Int; 97:1287-96), including the recent approval of ravulizumab by FDA and EMA.
   Several measures have been included in the study design to minimize biases associated with this single arm open-label design including primary and majority of secondary and efficacy endpoints that will be objectively measured via laboratory assessments (i.e. platelets, LDH, hemoglobin and creatinine).

A multicenter setting is chosen for the study to ensure adequate recruitment and representative enrollment of patients from a wide range of geographic regions for this rare indication.

Complete TMA response is a well-defined and accepted endpoint in clinical trials with aHUS and has been used in most recent clinical studies in aHUS patients (Fakhouri, et al. 2016, Rondeau, et al. 2020). It is designed to measure a simultaneous hematologic and kidney improvement in patients with TMA. Platelets and LDH are the most frequent variables used for evaluating treatment response in patients with aHUS (Rondeau, et al. 2020). Although no formal comparison analyses are planned for this study, results from iptacopan treated patients will be evaluated in the context of results reported in eculizumab (Fakhouri, et al. 2016) and ravulizumab (Rondeau, et al. 2020) studies. The calculated TMA response rate will be compared to a pre-defined threshold that has been chosen based on the two historical trials that are comparable in study design, population and efficacy endpoints (eculizumab (Fakhouri, et al. 2016) and ravulizumab (Rondeau, et al. 2020)). The TMA response rate and its 95% confidence interval based on asymptotic Gaussian approximation with continuity correction method for eculizumab and ravulizumab trials were 56.1% [39.7%, 72.5%] and 53.6% [39.6%, 67.5%], respectively. Given the single arm nature of historical trials, it is difficult to hypothesize the actual extent of eculizumab or ravulizumab effect versus placebo. However, the lower boundaries of 95% confidence interval (˜40%) could be considered as a demonstrated effect over placebo and taken as the reference. A 30% threshold has been chosen to ensure the preservation of ˜75% of this reference. That also corresponds to the preservation of >50% of the treatment effect when considering the point estimates from the historical trials.

Secondary efficacy endpoints include key hematological and kidney parameters that are clinically important to aHUS prognosis, including changes in eGFR and CKD status, dialysis requirement during treatment, as well as improvements in hematological parameters (hemoglobin).

The screening period ensures differential diagnosis of aHUS and also ensures that all participants have been appropriately vaccinated. Following 26 weeks of the core treatment period, participants will continue to the 26 weeks Extension Treatment period with a monthly follow-up. The core treatment duration of 26 weeks is considered appropriate to assess the effect of iptacopan on the primary and secondary efficacy endpoints as well as safety and tolerability of iptacopan, and identical study treatment duration has been previously studied in aHUS patients (Fakhouri, et al. 2016, Rondeau, et al. 2020). The Extension Treatment period of 26 weeks will provide long-term safety data and efficacy data on iptacopan in aHUS.

The study population will include adult patients diagnosed with aHUS and who are treatment naive to complement inhibitor therapy. Patients will have a diagnosis of aHUS with evidence of TMA including low platelet count (<150×10⁹/L), microangiopathic hemolytic anemia (LDH ≥1.5×ULN, hemoglobin ≤LLN) and decreased kidney function (serum creatinine ≥ULN).

A Patient Selection Committee has been established to review patient's eligibility and confirm patient's enrollment into the study. As the study is running in multiple centers worldwide where clinical practice may differ, the Committee will ensure an independent review of the aHUS diagnosis of each patient in this global study, thereby the Committee will standardize any geographical differences which may exist in diagnosing primary aHUS.

Genetic testing will be done for a selected set of genes known to be involved in aHUS etiology as it provides important prognostic information related to aHUS such as study treatment response, relapse and recurrence after transplantation. However, this aHUS specific genetic analysis will not be part of the screening process or determining eligibility. Additional assessments will include aHUS related biomarkers (such as C3, C4) and autoantibodies to complement proteins (such as factor H autoantibodies). Once clinical diagnosis of aHUS is confirmed by the investigator, genetic and biomarkers/auto-antibody testing will be performed wherever permitted per local regulations and after specific consent has been obtained from the patient.

### Dose and Duration Rationale

Iptacopan at 200 mg b.i.d. (wherein the dosing amount refers to the anhydrous free base of iptacopan) has been selected for this study based on the totality of safety, efficacy and favorable benefit-risk ratio data from the first in human (FIH) studies and the Phase 2 studies in C3 Glomerulopathy (C3G), paroxysmal nocturnal hemoglobinuria (PNH), and IgA nephropathy (IgAN).

In the FIH studies, there was rapid suppression of the AP activity (Wieslab) with approximately 80% or greater inhibition achieved at two hours post-dose for participants receiving single dose of 200 mg iptacopan (wherein the dosing amount refers to the anhydrous free base of iptacopan). The suppression of the AP (80% or greater inhibition) was sustained over 14 days of dosing at 200 mg b.i.d (wherein the dosing amount refers to the anhydrous free base of iptacopan). The exposure-response model developed with data from the FIH study with iptacopan in healthy volunteers predicts that a dose of about 200 mg b.i.d (wherein the dosing amount refers to the anhydrous free base of iptacopan) would be needed to achieve >90% inhibition of the AP (Wieslab assay) in most participants. Preclinical studies supported acceptable safety margins for human exposure following 200 mg b.i.d. dosing (wherein the dosing amount refers to the anhydrous free base of iptacopan).

Given the need for rapid and sustained AP inhibition in patients with aHUS and the observed PK/PD profile in a first in human study in healthy volunteers, a 200 mg b.i.d dose of iptacopan is selected for this study (wherein the dosing amount refers to the anhydrous free base of iptacopan).

In the human absorption, distribution, metabolism, and excretion (ADME) study in healthy volunteers, direct kidney excretion of iptacopan accounted for 20-25% of overall iptacopan dose, with the feces being the major route of excretion (>71%). With a limited kidney contribution to the overall iptacopan clearance (20-25%), the expected increase in iptacopan exposure in patients with kidney insufficiency would be minor and likely within the variability in exposure seen across patients in different iptacopan studies.

Core treatment duration of 26 weeks was chosen to make the study comparable to recent studies in aHUS patients with eculizumab (Fakhouri, et al. 2016) and ravulizumab (Rondeau, et al. 2020). Total study treatment duration of 52 weeks will enable assessment of long-term safety and efficacy in aHUS patients.

### Screening

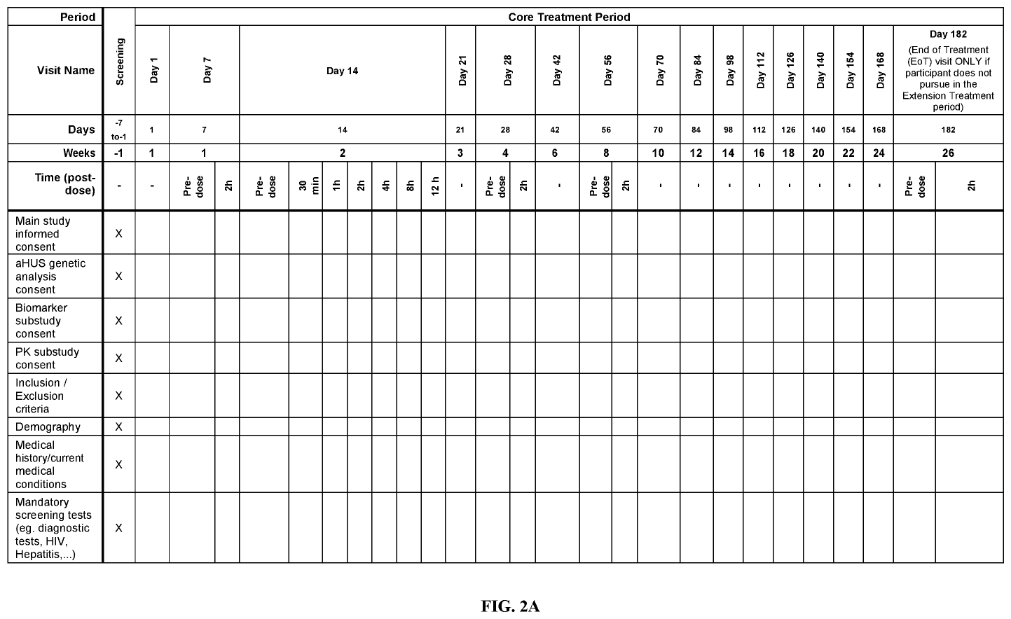

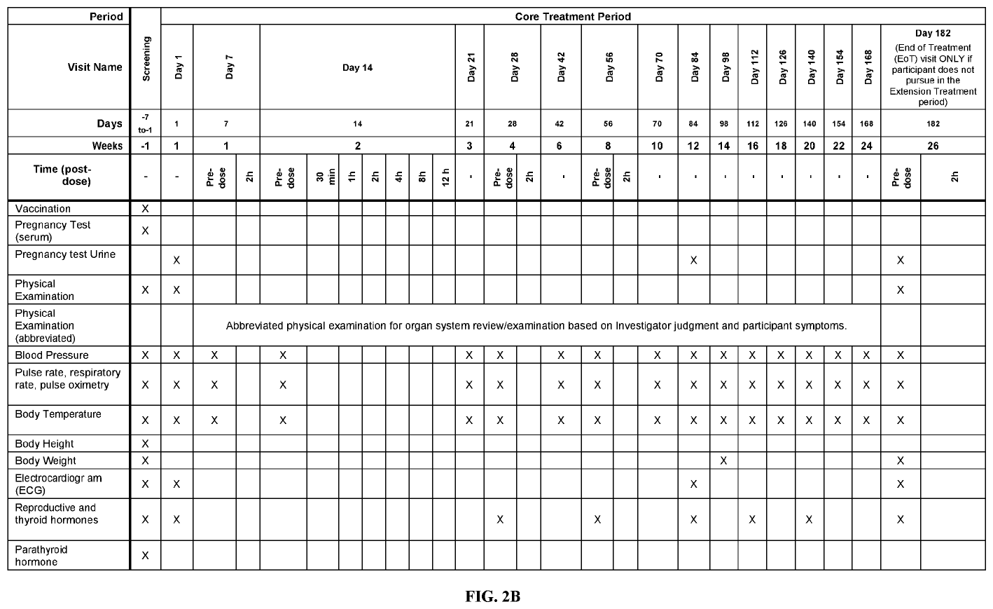

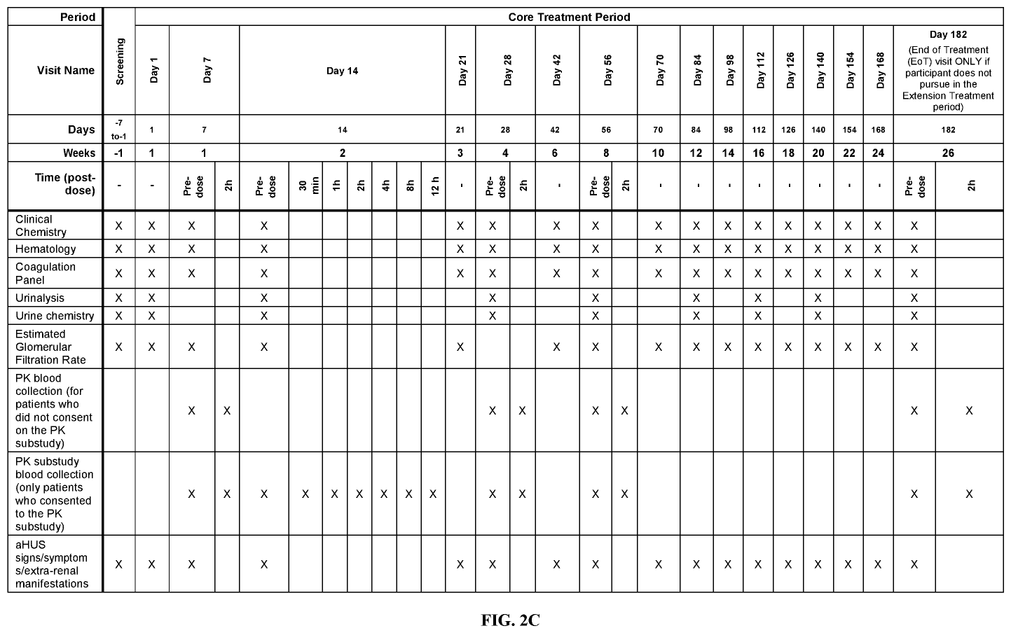

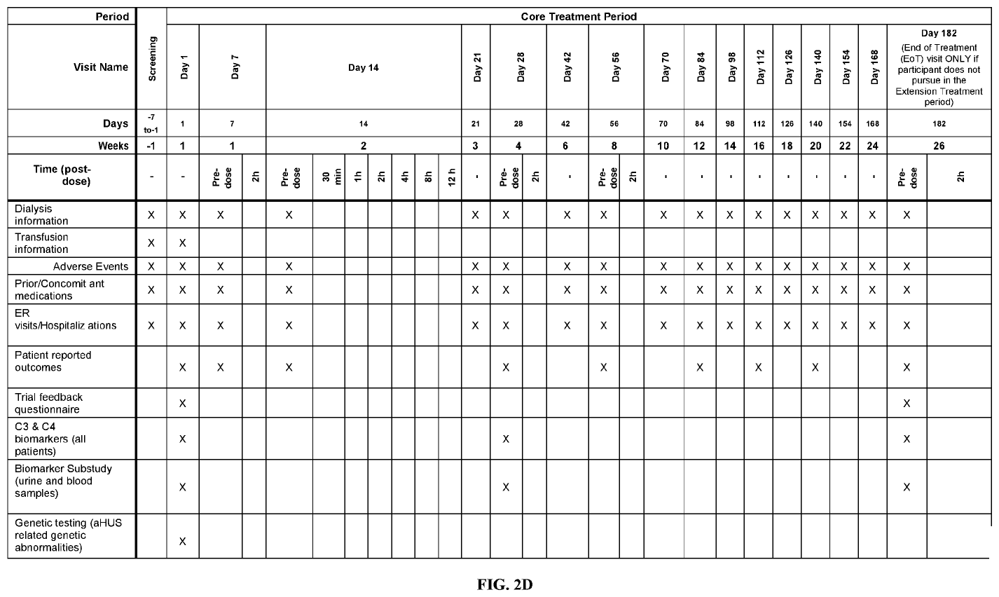

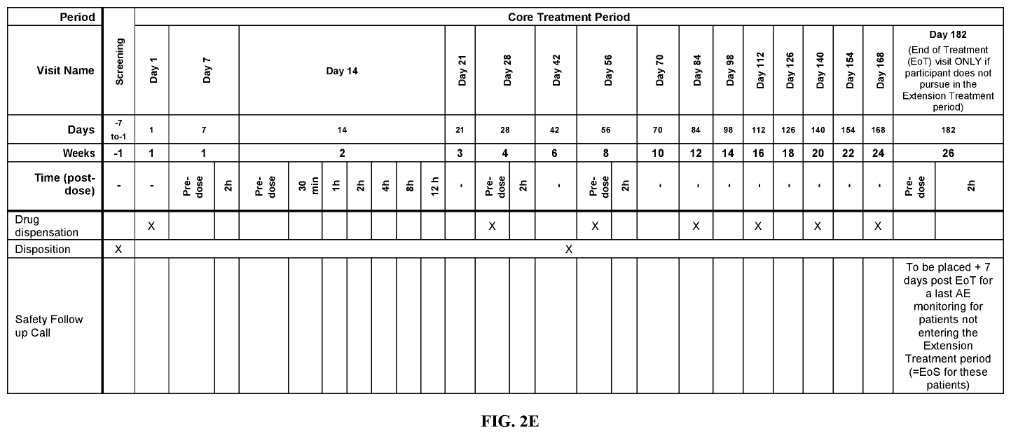

The screening visit's assessments will be followed as outlined as shown in FIGS. 2A-2E, and as applicable: participants who have not received the required vaccinations should be vaccinated. Vaccines should cover as many serotypes as possible (including meningococcal serotypes A, C, Y, W-135 and B). To minimize patient burden, the use of multivalent vaccines is recommended as locally available and per local guidelines and regulations (e.g. quadrivalent vaccine for N. meningitidis, which covers serotypes A, C, Y and W-135 and Pneumovax-23 which covers 23 S. pneumoniae serotypes). For the vaccination type and booster requirements use local guidelines, and locally available vaccines (and refer to the package insert).

Vaccinations should be started as early as possible. Patients who have not been vaccinated prior to initiating iptacopan study treatment should receive appropriate prophylactic antibiotics prior to and for at least 2 weeks after vaccination. If eligibility criteria are not met, the study participant should be considered as having failed screening and should not proceed further. The study participant can be re-screened.

### Core Treatment Period

Participants who are confirmed to meet the eligibility criteria will proceed to the open-label core treatment period. Treatment with iptacopan at a dose of 200 mg b.i.d. (wherein the dosing amount refers to the anhydrous free base of iptacopan) will start on the first day (Day 1) and continue for 26 weeks with study visits and corresponding assessments according to the schedule described in FIGS. 2A-2E.

Because of the known increased risk of infections with encapsulated bacteria, all participants will be provided with a Patient Safety Card. Participants will be instructed to be vigilant for any clinical sign of bacterial infections and to contact the investigator or local physician immediately in case of suspicion of infection. If indicated, antibiotic treatment should be started as soon as possible.

The use of PE/PI and anti-C5 antibody to treat aHUS is not allowed once study participants start study treatment with iptacopan in the study. Dialysis will be allowed during the study based on medical need as judged by the investigator. It is recommended that dialysis occurs at least 2 hrs after intake of iptacopan.

Participants who discontinue iptacopan study treatment administration during the core treatment period should not discontinue from the study (unless consent is withdrawn), but complete all visits and assessments up to Week 26 visit of the core treatment period. For these patients, the Week 26 (Day 182) visit assessments and the 7 days-post-EoT safety follow up phone call should be performed as End of Study (EoS) visit/assessments for the trial as they will not pursue in the Extension Treatment phase of the study.

The core treatment period will end with the completion of the Week 26 (Day 182) visit assessments. In the event that a study participant withdraws consent at any time during the core treatment period, a last visit shall be performed to record patient's withdrawal (i.e. Week 26 (Day 182) visit assessments should be performed as End of Study (EoS) visit for the trial).

### Extension Treatment Period

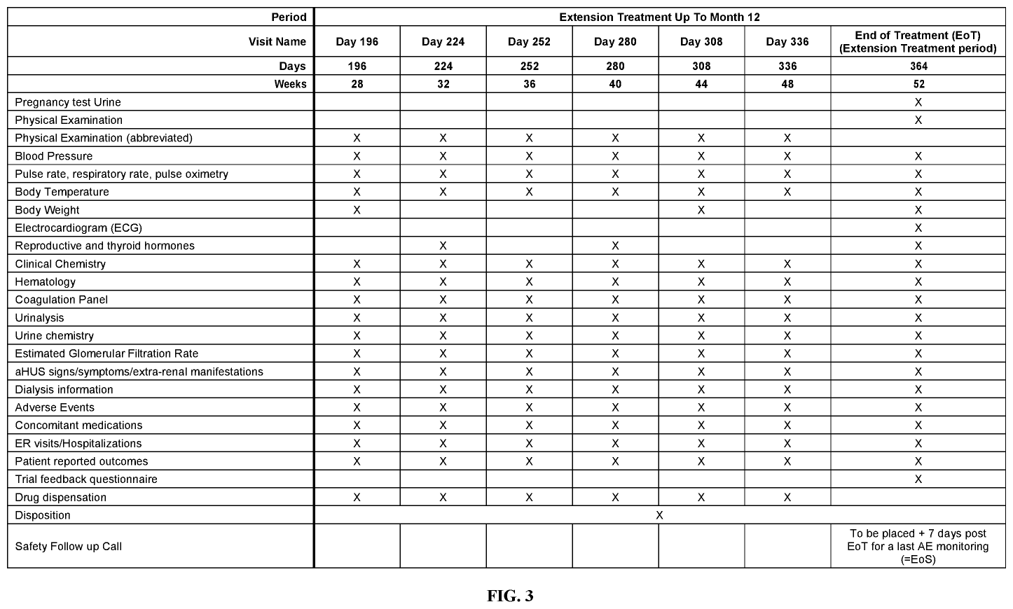

After completion of the 26 weeks core treatment period, study participants will continue study treatment with iptacopan and enter the Extension Treatment period of 26 weeks. The study visits and assessments detailed in FIG. 3 will be followed.

### Treatment Assignment

No randomization will be performed in this study; all eligible participants will receive open-label iptacopan 200 mg b.i.d. treatment (wherein the dosing amount refers to the anhydrous free base of iptacopan).

### Study Population

The study will enroll patients ≥18 years of age, diagnosed with aHUS and who have never been treated with complement inhibitor therapy, including anti-C5 antibody. A total of approximately 50 participants will be starting study treatment in the trial. The study will aim for approximately 5 patients treated with iptacopan with prior kidney transplantation.

### Inclusion Criteria

Participants eligible for inclusion in this study must meet all of the following criteria:
1. Signed informed consent must be obtained prior to participation in the study.
2. Willing and able to comply with the study visit schedule.
3. Male and female participants ≥18 years of age at the time of consent.
4. Evidence of thrombotic microangiopathy (TMA), including thrombocytopenia, evidence of hemolysis, and kidney injury, based on the following laboratory findings:
   Platelet count <150×10⁹/L during the Screening Period or within 28 days prior to the start of the Screening Period, and
   LDH ≥1.5×upper limit of normal (ULN) during the Screening Period or within 28 days prior to the start of the Screening Period and hemoglobin ≤lower limit of normal (LLN) for age and gender during the Screening Period or within 28 days prior to the start of the Screening Period, and
   Serum creatinine ≥ULN during the Screening Period. Patients requiring dialysis for acute kidney injury are eligible.
2. Vaccination against Neisseria meningitidis infection is required prior to the start of study treatment. If the patient has not been previously vaccinated, or if a booster is required, vaccine should be given according to local regulations, at least 2 weeks prior to first study drug administration. Patients who have not been vaccinated prior to initiating iptacopan treatment should receive prophylactic antibiotics prior to and for at least 2 weeks after vaccination.
3. If not received previously, vaccination against Streptococcus pneumoniae and Haemophilus influenzae infections should be given, according to local regulations. The vaccines should be given at least 2 weeks prior to first study drug administration. Patients who have not been vaccinated prior to initiating iptacopan treatment should receive prophylactic antibiotics prior to and for at least 2 weeks after vaccination.
4. Among patients with a kidney transplant
   Known history of aHUS prior to current kidney transplantation, or
   No known history of aHUS, and persistent evidence of TMA at least 4 days after modifying the immunosuppressive regimen (eg, suspending or reducing the dose) of calcineurin inhibitor ([CNI]; eg, cyclosporine, tacrolimus) or mammalian target of rapamycin inhibitor ([mTORi]; eg, sirolimus, everolimus).

### Exclusion Criteria

Participants meeting any of the following criteria are not eligible for inclusion in this study:
1. Treatment with complement inhibitors, including anti-C5 antibody
2. ADAMTS13 deficiency (activity <5% confirmed by central laboratory). Eligibility for this diagnostic test may be defined using results from local laboratory done as standard of care for the current TMA.
3. Shiga toxin-related hemolytic uremic syndrome (STX-HUS) associated with shiga-toxin producing bacteria such as Escherichia coli or Shigella dysenteriae, as demonstrated by a positive test for Shiga toxin. Eligibility for these diagnostic tests may be defined using results from local laboratory done as standard of care for the current TMA.
4. Positive direct Coombs test. Eligibility for this diagnostic test will be confirmed using results from local laboratory for tests done as standard of care for the current TMA.
5. Identified drug exposure-related HUS
6. HUS related to known genetic defects of cobalamin C metabolism
7. Known diacylglycerol kinase & (DGKE) mediated aHUS
8. Hemolytic condition with glucose-6-phosphate dehydrogenase (G6PD) deficiency
9. Bone marrow transplantation (BMT)/hematopoietic stem cell transplantation (HSCT)
10. Systemic sclerosis (scleroderma), systemic lupus erythematosus (SLE), or antiphospholipid antibody positivity or syndrome
11. Chronic hemo- or peritoneal dialysis (defined as maintenance dialysis as kidney replacement therapy for kidney failure)
12. Receiving PE/PI, for 28 days or longer, prior to the start of screening for the current TMA
13. Heart, lung, small bowel, pancreas, or liver transplantation
14. In patients with a kidney transplant, acute kidney dysfunction consistent with the diagnosis of transplantation failure due to acute/chronic active T-Cell mediated rejection (TCMR) and/or active/chronic active antibody-mediated rejection (ABMR) according to Banff 2017 criteria (Haas M, et al. (2018) The Banff 2017 Kidney Meeting Report: Revised diagnostic criteria for chronic active T cell-mediated rejection, antibody-mediated rejection, and prospects for integrative endpoints for next-generation clinical trials. Am J Transplant; 18:293-307)
15. Among patients with native kidney, history of any kidney disease other than aHUS, such as:
   Known kidney biopsy finding suggestive of underlying disease other than aHUS
   Kidney ultrasound finding demonstrating small kidneys suggestive of longstanding renal failure
   Known family history and/or genetic diagnosis of non-complement mediated genetic renal disease (e.g., focal segmental glomerulosclerosis)
16. Abnormal results of coagulation panel at screening suggestive of Disseminated Intravascular Coagulation (DIC)
17. Liver disease or liver injury at screening:
   As indicated by abnormal liver function tests (any single parameter of alanine amino transferase (ALT), gamma-glutamyl transferase (GGT), alkaline phosphatase (ALP) must not exceed 3×ULN)
   Active hepatitis B or C infection defined as HBV positivity or HCV positivity at screening
18. Patients with sepsis, severe systemic infection or COVID-19 infection
19. Presence of systemic infections (bacterial, viral, fungal or parasitic) that, in the opinion of the Investigator, confounds an accurate diagnosis of aHUS or impedes the ability to manage the aHUS disease
20. Active infection, or history of recurrent invasive infections, caused by encapsulated bacteria, i.e. meningococcus, pneumococcus (including Streptococcus pneumoniae associated HUS) or H. influenzae
21. Human immunodeficiency virus (HIV) infection (known history of HIV or test positive for HIV at screening)
22. Use of other investigational drugs within 5 half-lives of enrollment, or within 30 days, whichever is longer; or longer if required by local regulations.
23. History of hypersensitivity to any of the study drugs or its excipients or to drugs of similar chemical classes
24. Ongoing drug or alcohol abuse that could interfere with patient's participation in the trial
25. Patients receiving chronic intravenous immunoglobulin (IVIg) within 8 weeks prior to the start of screening, unless for unrelated medical condition (eg. hypogammaglobinemia); or chronic rituximab therapy within 12 weeks prior to the start of screening
26. Patients receiving other immunosuppressive therapies such as steroids, mTORi (eg, sirolimus, everolimus), calcineurin inhibitors (eg, cyclosporine A or tacrolimus) are excluded unless:
   Part of an established post-transplant antirejection regimen, or
   Steroids are being used for a condition other than aHUS (eg, asthma bronchiale)
27. Use of tranexamic acid within 7 days prior to screening
28. History of malignancy of any organ system (other than localized basal cell carcinoma of the skin or in situ cervical cancer), treated or untreated, within the past 5 years
29. Major concurrent comorbidities including but not limited to advanced cardiac disease (e.g., NYHA class IV), severe pulmonary disease (e.g., severe pulmonary hypertension (WHO class IV)), or hepatic disease (e.g., active hepatitis) that in the opinion of the investigator precludes participant's participation in the study
30. Any medical condition deemed likely to interfere with the patient's participation in the study
31. In patients with onset of TMA postpartum (persistent evidence of TMA for >3 days after the day of childbirth), evidence of postpartum hemorrhage (estimated blood loss greater than 500 ml)
32. Pregnant or nursing (lactating) women
33. Women of child-bearing potential, defined as all women physiologically capable of becoming pregnant, unless they are using effective methods of contraception during dosing of investigational drug and for 1 week after stopping of investigational drug. Effective contraception methods include:
   Total abstinence (when this is in line with the preferred and usual lifestyle of the participant). Periodic abstinence (e.g., calendar, ovulation, symptothermal, post-ovulation methods) and withdrawal are not acceptable methods of contraception
   Female sterilization (have had surgical bilateral oophorectomy with or without hysterectomy), total hysterectomy or bilateral tubal ligation at least six weeks before taking investigational drug. In case of oophorectomy alone, only when the reproductive status of the woman has been confirmed by follow up hormone level assessment
   Male sterilization (at least 6 months prior to screening). For female participants on the study, the vasectomized male partner should be the sole partner for that participant
   Barrier methods of contraception: Condom or Occlusive cap (diaphragm or cervical/vault caps). For UK: with spermicidal foam/gel/film/cream/vaginal suppository
   Use of oral, (estrogen and progesterone), injected or implanted hormonal methods of contraception or other forms of hormonal contraception that have comparable efficacy (failure rate <1%), for example hormone vaginal ring or transdermal hormone contraception or placement of an intrauterine device (IUD) or intrauterine system (IUS)
      In case of use of oral contraception women should have been stable on the same pill for a minimum of 3 months before taking investigational drug.

Women are considered post-menopausal if they have had 12 months of natural (spontaneous) amenorrhea with an appropriate clinical profile (e.g. age appropriate, history of vasomotor symptoms). Women are considered not of child bearing potential if they are post-menopausal or have had surgical bilateral oophorectomy (with or without hysterectomy), total hysterectomy or bilateral tubal ligation at least six weeks ago. In the case of oophorectomy alone, only when the reproductive status of the woman has been confirmed by follow up hormone level assessment is she considered not of child bearing potential. If local regulations deviate from the contraception methods listed above to prevent pregnancy, local regulations apply and will be described in the ICF.

### Treatment

All participants starting study treatment in this single arm open label study will receive iptacopan 200 mg b.i.d. (wherein the dosing amount refers to the anhydrous free base of iptacopan). No other treatment beyond iptacopan is included in this trial (see Table 1 for details of investigational drug).

TABLE 1
Investigational Drug

| Investigational/ | Pharmaceutical | Route of | | Sponsor (global |
|---|---|---|---|---|
| Control Drug | Dosage Form | Administration | Presentation | or local) |
| Iptacopan, 200 | Hard gelatin | Oral use | Open label, | Sponsor (global) |
| mg | capsule | | patient specific | |
| | | | kits | |

### Treatment Duration

The planned duration of core treatment period is 26 weeks followed by an Extension Treatment period of 26 weeks. Participants may be discontinued from study treatment earlier due to unacceptable toxicity and/or study treatment is discontinued at the discretion of the investigator or the participant.

If a participant discontinues study treatment for any reason during the core treatment period, every effort must be made to continue with the study assessments up to Week 26.

In the event a participant discontinues study treatment during the Extension Treatment period for any reason, every effort must be made to continue with the study assessments up to Week 52.

### Prohibited Medication

Use of the treatments and procedures listed below are not allowed during iptacopan administration:
· Any other complement inhibitors, including anti-C5 antibody, are prohibited for the entire study treatment duration. If other complement inhibitors (e.g., anti-C5 antibody) is started, the patient should permanently discontinue iptacopan.
· Live vaccines are prohibited for the entire study treatment duration
· Concomitant use of plasma exchange/plasma infusion is prohibited for the entire study treatment duration.
· In aHUS patients with confirmed anti-complement factor antibodies, use of any immunosuppressive therapies (eg. corticosteroids, mycophenolate mofetil (MMF), rituximab) is not allowed during the core treatment period (first 26 weeks). After the core treatment period, immunosuppressive therapies can be used per investigator judgement.
· Cyclophosphamide is prohibited for the entire study treatment duration; if cyclophosphamide is started, the patient should permanently discontinue iptacopan.
· Preclinical studies have shown that systemic disposition of iptacopan is primarily mediated by metabolic clearance, predominantly by cytochrome P450 2C8 isoform (CYP2C8) and to a smaller extent by direct glucuronidation. In addition, some contribution from direct kidney (approximately 20%) and direct biliary excretion (around 5 to 10%) is anticipated. Iptacopan is also a substrate for the organic anion-transporting polypeptide (OATP) hepatic uptake transporter. To ensure participant safety, co-medications that inhibit multiple disposition mechanisms of iptacopan (e.g. Gemfibrozil) are prohibited. The same applies to strong CYP2C8 inhibitors (main clearance pathway) such as clopidogrel.
· Gemfibrozil (a potent inhibitor of metabolizing enzymes CYP2C8, UGT1A and liver uptake transporter OATP1B1) must be interrupted at least 48 hours before first iptacopan dose until end of iptacopan study treatment (and replaced with another appropriate medication used for that indication).
· Strong inhibitors of CYP2C8 such as clopidogrel must be interrupted 7 days before first iptacopan dose until end of iptacopan study treatment (and replaced with another appropriate medication).
· Medications that are either “sensitive substrates” for the efflux transporter P-gp or have a narrow therapeutic index (NTI) and are substrates for P-gp (should not be administered with iptacopan (interrupted 48 hours before first iptacopan dose). Typical examples are digoxin, quinidine, paclitaxel, fentanyl and phenytoin. However, if no alternative treatment is available, a staggered dosing approach is recommended (refer to Section 6.2.1.1).
· Concomitant medication listed under exclusion criteria 25 and 26 is prohibited except for the condition specified in the criterion.

### Visit Schedule and Assessments

The assessment schedules shown in FIGS. 2A-2E and 3 list which assessments as well as when they are performed. Participants who discontinue from iptacopan study treatment for any reason during the core treatment period should continue in the study up to Week 26 visit, completing all scheduled visits assessments. Participants who discontinue from iptacopan study treatment during the Extension Treatment period of the study for any reason should continue in the study up to the Week 52 visit completing all scheduled visit assessments.

### Efficacy

Efficacy/pharmacodynamic assessments will be collected at the time points defined in the Assessment schedules shown in FIGS. 2A-2E and 3.

### Complete TMA Response

The primary efficacy assessment is Complete TMA Response without the use of PE/PI and anti-C5 antibody during the 26-week core treatment period. The criteria for complete TMA response are the following:
· Hematological normalization in platelet count (platelet count ≥150×10⁹/L) and LDH (below ULN),
· Improvement in renal function (≥25% serum creatinine reduction from baseline),
   Patients must meet all Complete TMA Response criteria at two separate assessments obtained at least 4 weeks apart, and any measurement in between, to be classified as having met the primary efficacy endpoint.

### Hematology and Chemistry Parameters

Blood samples for hematology and clinical chemistry will be collected according to Assessment schedules shown in FIGS. 2A-2E and 3. The following laboratory parameters will be assessed for the efficacy endpoint: Hemoglobin, platelets and LDH.

Estimated Glomerular Filtration Rate (eGFR) and CKD Stage

Serum creatinine as measured in mg/dL as part of the clinical chemistry panel through the central laboratory will be used to calculate the eGFR applying the Chronic Kidney Disease Epidemiology Collaboration (CKD-EPI) formula (Levey A S, et al. (2009) A new equation to estimate glomerular filtration rate. Ann Intern Med; 150(9):604-12) for participants 18 years or older.

GFR=141×min(Scr/κ,1)^{α}×max(Scr/κ,1)^{-1.209}×0.993^{Age}×1.018 [if female]×1.159 [if black]

where: Scr is serum creatinine in mg/dL, κ is 0.7 for females and 0.9 for males, α is −0.329 for females and −0.411 for males, min indicates the minimum of Scr/κ or 1, and max indicates the maximum of Scr/κ or 1.

The equation does not require weight because the results are reported normalized to 1.73 m² body surface area, which is an accepted average adult surface area. Changes in CKD status will be assessed based on eGFR categories shown in Table 2.

TABLE 2
GFR categories in chronic kidney disease (CKD)

| | | eGFR |
|---|---|---|
| | | (ml/min/ |
| CKD stage | Description | 1.73 m²) |
| Stage 1 (G1) | Kidney damage with normal kidney function | ≥90 |
| Stage 2 (G2) | Mild loss of kidney function | 60-89 |
| Stage 3 (G3) | 3a: Mild to moderate loss of kidney function; | 3a: 45-59 |
| | 3b: Moderate to severe loss of kidney | 3b: 30-44 |
| Stage 4 (G4) | Severe loss of kidney function | 15-29 |
| Stage 5 | Kidney failure and need for transplant or | <15 |
| End stage | dialysis | |
| renal disease | | |
| (kidney failure) | | |

(Source: KDIGO 2012 Clinical Practice Guideline for the Evaluation and Management of Chronic Kidney Disease; 3: 19-62)

### Dialysis

The need for hemo- or peritoneal dialysis will be monitored by the investigator during the study. Information on any dialysis treatment performed for participants will be collected in case report form (CRF) as per the assessment schedules shown in FIGS. 2A-2E and 3.

Signs, Symptoms and Extra-Renal Manifestations of aHUS

aHUS signs and symptoms will be collected according to the assessment schedules shown in FIGS. 2A-2E and 3. The investigator (or designee) will record the presence of the following signs and symptoms in the CRF based on participants self-reporting or elicited by the investigator:
· Chest pain
· Shortness of breath/dyspnea
· Headache
· Confusion
· Irritability
· Anxiety
· Fatigue/feeling weak or tired
· Abdominal pain
· Nausea and/or vomiting
· Diarrhea
· Malaise
· Yellow discoloration of eyes and/or skin (jaundice)
· Easy bruising
· Swelling
   In addition, extra-renal manifestations of aHUS will be evaluated by the investigator (according to the assessment schedules shown in FIGS. 2A-2E and 3) using a composite of laboratory and clinical measurements (vital signs and an organ system review). These extra-renal manifestations will be reported in the CRF. Extra-renal manifestations may involve neurological, ocular, gastrointestinal, pancreatic, respiratory system or others.

### Appropriateness of Efficacy Assessments

Complete TMA response is a well-defined and accepted endpoint in clinical trials with aHUS and has been used in most recent clinical studies in aHUS patients (Fakhouri, et al. 2016, Rondeau, et al. 2020). It is designed to measure a simultaneous hematologic and renal improvement in patients with TMA. Platelets and LDH are the most frequent variables used for evaluating treatment response in patients with aHUS (Rondeau, et al. 2020). The efficacy assessments including laboratory parameters hemoglobin (to determine the degree of anemia), platelet count and LDH (as marker for hemolysis), changes in eGFR and CKD status and dialysis requirement during treatment are important parameters for assessing treatment response in aHUS.

Functional Assessment of Chronic Illness Therapy-Fatigue (FACIT-Fatigue), along with general quality of life instrument EQ-5D (EuroQol Group) and the Short form 36 health survey (SF-36) questionnaires were used previously to measure various aspects of fatigue in patients with aHUS (Fakhouri, et al. 2016, Greenbaum L A, et al. (2020) Functional Assessment of Fatigue and Other Patient-Reported Outcomes in Patients Enrolled in the Global aHUS Registry. Kidney Int Rep; 5:1161-71). In this study, quality of life will be assessed using PRO instruments FACIT-Fatigue, Patient Global Impression of Severity (PGIS), EuroQol 5-level EQ-5D version (EQ-5D-5L) and SF-36 v2.

### Pharmacokinetics

PK samples (pre-dose and 2 hr post dose) will be collected for all participants on Days 7, 28, 56 and 182 as indicated in Table 3. As no food effect has been observed in study CLNP023X2101 patients do not need to be fasted on days of PK analysis. In addition, a PK substudy will be conducted in some sites to characterize iptacopan exposure in aHUS patients. Up to 8 adult participants will be included in the PK substudy. For participants in the PK substudy, a 12-hour full PK profile will be taken on Day 14; in addition to the PK samples taken on Day 7, Day 28, Day 56 and Day 182 (Table 4).

TABLE 3
PK sample collection schedule for all patients

| | | | | | PK blood collection | | |
|---|---|---|---|---|---|---|---|
| | Visit | | | | Size | Dose | Sample |
| Period | Name | Days | Weeks | Time | (mL) | ref. ID | No. |
| Study | Day 7 | 7 | 1 | pre-dose | 2 | 1 | 101 |
| Treatment | | | | 2 h post- | 2 | 1 | 102 |
| Phase | | | | dose | | | |
| | Day 28 | 28 | 4 | pre-dose | 2 | 3 | 301 |
| | | | | 2 h post- | 2 | 3 | 302 |
| | | | | dose | | | |
| | Day 56 | 56 | 8 | pre-dose | 2 | 4 | 401 |
| | | | | 2 h post- | 2 | 4 | 402 |
| | | | | dose | | | |
| | Day 182 | 182 | 26 | pre-dose | 2 | 5 | 501 |
| | | | | 2 h post- | 2 | 5 | 502 |
| | | | | dose | | | |
| Unscheduled | Unscheduled | — | — | — | 2 | 6 | 1001+ |

TABLE 4
Sub-study PK sample collection schedule

| | | | | | PK blood collection | | |
|---|---|---|---|---|---|---|---|
| | Visit | | | | Size | Dose | Sample |
| Period | Name | Days | Weeks | Time | (mL) | ref. ID | No. |
| Study | Day 7 | 7 | 1 | pre-dose | 2 | 1 | 101 |
| Treatment | | | | 2 h post- dose | 2 | 1 | 102 |
| Phase | Day 14 | 14 | 2 | pre-dose | 2 | 2 | 201 |
| | | | | 0.5 h post- dose | 2 | 2 | 202 |
| | | | | 1 h post- dose | 2 | 2 | 203 |
| | | | | 2 h post- dose | 2 | 2 | 204 |
| | | | | 4 h post- dose | 2 | 2 | 205 |
| | | | | 8 h post dose | 2 | 2 | 206 |
| | | | | 12 h post- dose | 2 | 2 | 207 |
| | Day 28 | 28 | 4 | pre-dose | 2 | 3 | 301 |
| | | | | 2 h post- dose | 2 | 3 | 302 |
| | Day 56 | 56 | 8 | pre-dose | 2 | 4 | 401 |
| | | | | 2 h post- dose | 2 | 4 | 402 |
| | Day 182 | 182 | 26 | pre-dose | 2 | 5 | 501 |
| | | | | 2 h post- dose | 2 | 5 | 502 |
| Unscheduled | Unscheduled | — | — | — | 2 | 6 | 1001+ |

The number of samples/blood draws and total blood volume collected will not exceed those stated in the protocol. PK backup samples may be used for metabolism investigations or plasma protein binding in an exploratory setting as needed. Iptacopan will be determined by a validated LC-MS/MS method; the anticipated Lower Limit of Quantification (LLOQ) is 1.0 ng/mL. Concentrations will be expressed in mass per volume units (ng/ml) and will refer to the free base. PK parameters to be determined in the main study are Cmax (apparent) and Ctrough. In the full PK sub-study Cmax, Tmax, Ctrough and AUClast will be determined. Additional PK parameters will be calculated if feasible. Concentrations below the LLOQ will be reported as “zero” and missing data will be labeled as such in the Bioanalytical Data Report.

### Biomarkers

### Samples Collected in the Full Population

Blood will be collected in all participants on days 1, 28 and 182 as detailed in the Assessment schedule FIGS. 2A-2E to measure the complement related biomarkers C3 and C4. These results will be used to determine if baseline levels change with study treatment, predict participant outcomes and/or renal disease progression. Instructions for sample collection, processing, and shipment will be provided in the laboratory manual.

### Samples Collected in the Biomarker Substudy

As permitted by local governing regulations and Institutional Review Boards, it is required as part of this protocol that the Investigator presents the planned assessment of biomarkers/proteins to the participant. Blood and urine samples for these biomarker/protein assessments will be collected on days 1, 28 and 182 as detailed in the Assessment Schedule (Table 2A-2E).

Samples will be used to measure biomarkers or proteins related to complement pathways, aHUS, or iptacopan mechanism of action and include biomarkers such as Wieslab, Bb, both plasma and urinary sC5b-9. Sample for analysis of autoantibodies to complement proteins (eg. Factor H) will be collected once on day 1. Analysis is also planned to examine the effect of iptacopan on protein expression and may support the identification of biomarker signatures that characterize disease development and the response of treatment with iptacopan. In the event promising biomarkers/proteins (or methods) are discovered or new information on aHUS or study drug biomarker effect is obtained, additional analysis may be performed on remaining samples.

aHUS Genetic Panel

The study includes a genetic component to test for known genetic mutations associated with aHUS. Patients (or legal guardians) who decline from providing a sample for aHUS genetic testing can still participate in the study. As permitted by local governing regulations and by IRB/EC, it is required as part of this protocol that the Investigator presents this option to the participant.

The purpose of this genetic analysis is to better understand the patient population treated with iptacopan, and to better characterize the safety and efficacy of iptacopan in patients with aHUS with different underlying mutations. Approximately 60% of patients with aHUS have genetic or acquired abnormality in regulators of the AP leading to either low levels or functionally ineffective regulators (Noris M, et al. (2015) Glomerular Diseases Dependent on Complement Activation, Including Atypical Hemolytic Uremic Syndrome, Membranoproliferative Glomerulonephritis, and C3 Glomerulopathy: Core Curriculum 2015. Am J Kidney Dis; 66(2):359-75). The prognosis of aHUS depends on the underlying genetic abnormality, with poorer outcomes and relapses associated with specific mutations (Fremeaux-Bacchi, et al. 2013, Schaefer, et al. 2018).

As technology changes over time, the most appropriate technology will be used at the time this analysis is performed. This analysis will only test for the aHUS associated genes listed in Table 5. One time sampling for this analysis will be performed on Day 1 as detailed in FIGS. 2A-2E. Laboratory manuals will be provided with detailed information on sample collection, handling, and shipment. To maximize confidentiality, all samples will be double-coded to prevent the exposure of the participant's information and identity.

TABLE 5
Genes planned in the aHUS genetic testing

| Period | Visit | aHUS related genes |
|---|---|---|
| Study Treatment Phase | Day 1 | C3, CD46 (MCP), CFB, CFH, |
| | | CFHR1, CFI, DGKE, MMACHC |

C3, complement component 3; CD46, cluster of differentiation 46; MCP, membrane cofactor protein; CFB, complement factor B; CFH, complement factor H; CFHR, complement factor H-related protein; CFI, complement factor I; DGKE, diacylglycerol kinase epsilon; MMACHC, methylmalonic aciduria and homocystinuria type C protein

### Study Discontinuation and Completion

Discontinuation of study treatment for a participant occurs when study treatment is permanently stopped for any reason (prior to the planned completion of study treatment administration, if any) and can be initiated by either the participant or the investigator.

Study completion is defined as when the last participant finishes their End of Study visit and any repeat assessments associated with this visit have been documented and followed-up appropriately by the Investigator or, in the event of an early study termination decision, the date of that decision. Participants who complete the study may be eligible to enroll in a single-arm open-label iptacopan rollover extension program (REP). Participants who prematurely withdraw from the study for any reason are not eligible to enroll in the REP.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A use of iptacopan or a pharmaceutically acceptable salt thereof, for the treatment of atypical hemolytic uremic syndrome (aHUS) in a subject, wherein the treatment comprises orally administering to the subject a therapeutically effective amount of iptacopan or a pharmaceutically acceptable salt thereof.

2. The use of claim 1, wherein the treatment comprises orally administering to the subject a therapeutically effective amount of iptacopan hydrochloride.

3. The use of claim 1 or 2, wherein the therapeutically effective amount is 200 mg based on an amount of anhydrous free base of iptacopan.

4. The use of any one of claims 1 to 3, wherein the treatment comprises orally administering to the subject a therapeutically effective amount of iptacopan or a pharmaceutically acceptable salt thereof twice daily (b.i.d.).

5. The use of any one of claims 1 to 4, wherein the subject is naive to complement inhibitor therapy.

6. The use of any one of claims 1 to 5, wherein the subject has been vaccinated against one or more of Neisseria meningitidis, Streptococcus pneumoniae, and Haemophilus influenzae infections.

7. The use of any one of claims 1 to 6, wherein the treatment comprises achieving complete thrombotic microangiopathy (TMA) response within 24 weeks after initiation of treatment.

8. The use of any one of claims 1 to 7, wherein achieving complete TMA response comprises achieving hematological normalization in platelet count within 24 weeks after initiation of treatment.

9. The use of any one of claims 1 to 8, wherein the platelet count is normalized after treatment with iptacopan or a pharmaceutically acceptable salt thereof to a range of about 150×10⁹/L to about 450×10⁹/L.

10. The use of any one of claims 1 to 9, wherein achieving complete TMA comprises achieving normalization of the level of lactate hydrogenase (LDH) after 24 weeks after initiation of treatment.

11. The use of claim 10, wherein the LDH level is reduced to below the upper limit of normal.

12. The use of any one of claims 1 to 11, wherein achieving complete TMA response comprises improving kidney function in the subject, comprising reducing serum creatinine levels by about 10% or more, as compared to a baseline.

13. The use of any one of claims 1 to 12, wherein the treatment comprises increasing the hemoglobin level in the subject by about 0.5 g/dL or more as compared to a baseline.

14. The use of any one of claims 1 to 13, wherein the treatment comprises stabilizing or improving the estimated glomerular filtration rate (eGFR) in the subject as compared to a baseline.
